(19) United States
(12) Patent Application Publication
(10) Pub. No.: US 2007/0037223 A1
(43) Pub. Date: Feb. 15, 2007

(54) ISOTOPE LABELING METHODS

(75) Inventors: Isao KANEKO, Osaka (JP); Megumu Kondo, Osaka (JP); Atsushi Miyachi, Osaka (JP); Masayuki Yokota, Osaka (JP)

Correspondence Address: SUGHRUE MION, PLLC, 2100 PENNSYLVANIA AVENUE, N.W., SUITE 800, WASHINGTON, DC, 20037, US

(73) Assignee: Japan Health Sciences Foundation and Director General of National Institute of Health Sciences

(21) Appl. No.: 11/379,761

(22) Filed: Apr. 21, 2006

(30) Foreign Application Priority Data
Jul. 14, 2005 (JP) 2005-205749

(51) Int. Cl. G01N 33/53

(52) U.S. Cl. 435/7.5

(57) ABSTRACT

The present invention relates to a method for the analysis of differential expression of proteins employing a radioactive label, characterized by cleaving a tag from peptides labeled with a cICAT reagent, separating and purifying the resultant labeled peptides, and performing an analysis in mass spectrometry.

### BACKGROUND OF THE INVENTION

1. Technical Field of the Invention

The present invention relates to an isotope labeling method for the analysis of differential expression of proteins. Specifically, the present invention relates to an improved method for performing an analysis of differential expression of a plurality of small-amount proteins in samples employing an ICAT reagent containing a cleavable tag (which hereinafter is simply referred to at times as a “cICAT reagent”), and to a system for such an analysis.

2. Description of Related Art

Genome analysis has actively been conducted in connection with diseases and aging and gives rise to a lot of results. Recently, further advancing of the analysis has made attempts to analyze a population of proteins which are expression products of genes in diseased or aging tissues and normal tissues (proteosome), thereby to identify proteins involved in diseases and aging. Various methods for the analysis of differential expression have been developed and are used for analyzing these proteosomes. It is on isotope labeling methods that attention is focused among them.

Isotope labeling method are an analytical method by which two types of isotope-labeled reagents that specifically react with amino acids or others in a protein (light- and heavy-chain labeled reagents designed to have a difference only in mass number employing an isotope) are used to separately label respective proteins to be compared, followed by trypsin treatment or the like, and the resulting peptides are subjected to measuring the ratio of amounts of light- and heavy-chain labeled peptides on a mass spectrometer, thereby to quantitatively examine differential expression of proteins. It is likely that these methods can be employed to identify proteins associated with diseases, for example, by performing an analysis of differential expression between proteins from patients and healthy individuals.

There are provided ICAT reagents as means for improving quantitativity, reproducibility, and other properties in these isotope-labeling methods. A cICAT reagent, which is a type of isotope-labeled reagents that specifically react with particular sites in a protein, is designed such that its segment contains a tag and labeled peptides containing the tag can be purified specifically, for example, on affinity columns, and in addition, the tag moiety can be cleaved from the labeled peptides, for example, with acid treatment (Hansen, K. C. et al., Mol. Cell Proteomics, 2:299-314, 2003). For example, there is a known routine procedure which employs a cICAT reagent using biotin as the tag (ABI protocol), and there are many reports saying that this protocol is effective in making a precise analysis of differential expression of many proteins in a variety of tissues and cells (T. Toda, et al., Eds., In Frontier of Disease Proteomics Idenshi, Igaku MOOK 2 (ISSN 1349-2527), pp. 233-243, 2005 (published by Medical Do), in Japanese). However, there have been few reports on the results of analyses of differential expression of proteins, which were performed in accordance with the above-described routine procedure, in samples, such as serum, having a plurality of small-amount proteins. Only twenty to thirty of serum proteins were identified and quantified (Zieske, L. R. et al., ASMS 2003, Poster Number W-032).

As mentioned above, isotope labeling methods utilizing cICAT reagents which are previously known are not always effective when making an analysis of differential expression of proteins in samples having a plurality of small-amount proteins, and thus there is great need of methods which are more effective for the analysis of differential expression. A purpose of the present invention is to provide, by improving an isotope labeling method employing a cICAT reagent, a method which effectively makes an analysis of differential expression of a plurality of small-amount proteins present in a sample, and is to provide a system therefor.

### SUMMARY OF THE INVENTION

The present inventors have made extensive studies in view of the above-described circumstances, and in consequence have found that samples in which, according to a routine procedure, serum samples were treated with a cICAT reagent and labeled peptides containing the tag were fractionated and purifying, followed by tag cleaving treatment of the obtained tag-containing sub-fractions, contained large amounts, which were not expected, of the tag and tag-containing byproducts derived from the reagent (which are collectively referred to as the “tag and others”), and these remaining tag and others are responsible for significantly reducing the number of serum proteins to be identified and quantified. Thus, the inventors modified the routine procedure and in consequence, have found that it is possible to perform an analysis of a much larger number of small-amount proteins than with the routine procedure, when the tag portion of the cICAT labeled peptides is cleaved in advance and the resulting sample is loaded on a column to move the remaining tag and others, followed by analyzing, on a mass spectrometer, the labeled peptides obtained by the separation and purification of the labeled peptides, leading to the completion of the invention.

Therefore, the present invention provides the following:

(1) a method for the analysis of differential expression of proteins employing isotope labeling, characterized by cleaving a tag from peptides labeled with a cICAT reagent, separating and purifying the resultant labeled peptides, and performing an analysis in mass spectrometry;

(2) the method according to (1), wherein the step of separation and purification is carried out using column chromatography and wherein the removal of the tag and others and the separation and purification of the cICAT-labeled peptides are carried out concurrently;

(3) the method according to (1) or (2), wherein the tag is biotin;

(4) the method according to any one of (1) to (3), wherein the peptides are derived from serum proteins;

(5) a system for performing an analysis of differential expression of small-amount proteins in a sample, characterized by employing a method according to any one of (1) to (3); and

(6) the system according to (5), wherein the sample is a serum sample.

According to the present invention are provided methods and systems enabling one to perform an efficient analysis of differential expression of a plurality of small-amount proteins in samples. Such methods can be used, for example, to make an analysis of differential expression between serum proteins from patients and healthy individuals, which has utility, for example, in searching proteins associated with diseases, and other applications.

The present invention, which provides methods and systems enabling one to perform an efficient analysis of differential expression of a plurality of small-amount proteins present in samples, can be used in the fields of proteomics studies, analytical instruments, and others,

### BRIEF DESCRIPTION OF THE DRAWINGS

FIG. 1 shows biotin fractions from a sample in which biotin-bound, cICAT-labeled serum peptides have been TFA treated and a fraction pattern by an SCX column chromatography of the cICAT-labeled serum peptides having the biotin removed therefrom. The peak of the biotin can be seen at a retention time of about 5 minutes and the peak of biotin-containing byproducts derived from the reagent at a retention time of about 14 minutes, demonstrating that the separation of the peptide peaks from the byproduct peak has been achieved.

FIG. 2 shows a Venn diagram representation of top 119 human-serum proteins identified by Q-Star XL and by ABT-4700.

FIG. 3 shows a Venn diagram representation of all the 311 human-serum proteins identified by Q-Star XL and by ABI-4700.

### DESCRIPTION OF PREFERRED EMBODIMENTS

The present invention will now be described in detail and unless otherwise explained, the terms as used herein are intended to have the meaning usually that are understood in the art.

The present invention, in a first aspect, provides a method for the analysis of differential expression of proteins employing isotope labeling, characterized by cleaving a tag from peptides labeled with a cICAT reagent, separating and purifying the resultant labeled peptides, and performing an analysis in mass spectrometry. Protein containing samples which can be subjected to the method according to the present invention are not limited in particular, and any sample may be used, including samples derived from animals, plants, and microorganisms. Examples of protein containing samples derived from animals include samples of body fluids obtained from mammals, particularly, from humans, such as serum, saliva, urine, sweat, and others. Examples of samples derived from plants include fruit juices, extracts of stems and leaves, extracts of seeds, extracts of underground stems, and others. Samples derived from microorganisms include various fermentations, cultures, microbial homogenates, and others. The present invention can be applied to these samples containing proteins, thereby to enable one to made an analysis of differential expression of the proteins, so as to investigate metabolic mechanisms of organisms, including animals, plants, and microorganisms. In particular, the present invention can be used to carry out proteomic studies, for example, for the identification of proteins associated with animal diseases and aging, or alternatively, for example, to make a diagnosis or examination of diseases in animals, including humans. As demonstrated in Examples, the present invention displays its power, especially in the analysis of differential expression of a wide variety of small-amount proteins in serum.

In the present method for the analysis of differential expression of proteins, protein containing samples described above are first treated with a cICAT reagent to obtain cICAT-labeled proteins. Conditions for the reaction of the cICAT reagent and the proteins contained in a sample will be varied, depending on the type of amino acids in the proteins to be labeled and the properties of the cICAT reagent. In general, the cICAT reagent is comprised of a site at which the reagent binds to a protein (for example, a site at which the reagent binds to cysteine of a protein), an isotope-labeled linker, a tag-cleaving site, and a tag. Binding of the cICAT reagent and a protein is usually covalent. As the isotope, various isotopes can be used, and stable isotopes are preferable. For example, combinations of ¹H and ²D, ¹²C and ¹³C, and others are employed. It may be possible that a sample from normal tissues is labeled with a ¹²C-containing cICAT reagent and a sample from diseased tissues is labeled with a ¹³C-containing cICAT reagent, thereby to perform an analysis of differential expression of proteins. As the tag, tags of any type can be used if their attachment facilitates the separation and purification of peptides and does not exert detrimental effects on the analysis of peptides, and include, for example, sugar containing groups, and others. Biotin is preferably used as the tag, because of easy and specific purification by use of avidin affinity chromatography. As the tag-cleaving site, sites of any type can be used if the tag can be cleaved with ease and without exerting detrimental effects on the labeled peptides. For example, use is usually made of tags which can be cleaved easily with acid treatment, such as TFA (trifluoroacetic acid).

It is well known in the art that an “ICAT” reagent stands for “Isotope-Coded Affinity Tags.” In the specification, an ICAT reagent containing a cleavable tag is referred to as a cICAT reagent, as described above. As cICAT reagents for use in the present invention are included various reagents, and they are commercially available. Typically, there is a Cleavable ICAT reagent from ABI employing biotin as the tag, which is preferably used in the present invention. The “Cleavable ICAT” is the registered trade name of ABI.

After the reaction of the proteins in a sample and the cICAT reagent, the resulting cICAT-labeled proteins are subjected to proteolysis to obtain cICAT-labeled peptides. This proteolysis can be carried out in various ways. For example, acid hydrolysis, enzymatic hydrolysis, and others can be utilized, Preferably, enzymatic hydrolysis is employed. Preferable proteolytic enzymes include trypsin, pepsin, and others, and trypsin is used more preferably.

After that, the tag portion is cleaved from the cICAT-labeled peptides obtained as described above. The cleavage of the tag at this stage is a feature of the present invention. In order to concentrate the cICAT-peptides and remove the contaminating materials, the cICAT-labeled peptides may be purified prior to the tag cleavage. To this end, it is usual to employ affinity chromatography using a substance which can specifically bind to the tag. For example, when the tag is biotin, column chromatography using a resin to which avidin has been bound can be performed, thereby to collect the cICAT-labeled peptides. Methods for cleaving the tag portion from the cICAT-labeled peptides will be varied, depending on the structure of the cICAT reagent, in particular, the type of tags, the class of analytes, and others. The cleavage reaction must be carried out under conditions exerting no effect on the peptides to be analyzed. For example, in the case of using a Cleavable ICAT reagent from ABI, TFA can be employed to cleave the biotin tag.

In the case when a subsequent step of separation and purification is carried out without the cleavage of the tag at such a stage as described above (i.e. in the case of conventional procedures, for example, when the ABI protocol is used), large amounts of the tag and others remain in the obtained sample, resulting in significant interference in the identification and quantification of proteins, especially small-amounts proteins. Moreover, conventional procedures require applying tag cleavage treatment to each of the peptide fractions obtained from the step of separation and purification and then carrying out the step of mass spectrometry, and thus take much time and labor. In contrast, the method of the present invention is free from these disadvantages and allows an efficient identification and quantification of a wide variety of small-amount proteins in a sample.

Subsequently, samples of the labeled peptides obtained by cleaving the tag according to the method of the present invention are subjected to the step of separation and purification. Although the step of separation and purification can be carried out using various procedures, it is preferable that column chromatography is employed so that the removal of the tag in the sample and the separation and purification of the peptides are carried out concurrently. Various supports for chromatography are commercially available and can be selected as appropriate, depending on the type of tags and analytes. For example, silica gel-based supports may be used, or SCX supports (poly-LC-sulphoethyl A supports) may be used, or supports having affinity for avidin (when the tag is biotin) may be used. Column conditions for elution will be determined as appropriate, depending on the properties of analytes and tags, and others. It may be effective to employ salt concentration gradient elution methods. It is preferable in terms of resolution, rapidity, and others that column chromatography is carried out using HPLC. In addition, the step of separation and purification is not limited to the use of columns, and methods of using filters, batch processes, and others can be employed. Such a step of separation and purification may be carried out twice or more. Furthermore, samples may be concentrated before subjecting them to the step of separation and purification. In general, chromatograms are recorded and fractions corresponding to respective peaks are pooled in the step of separation and purification. Each of the fractions can be desalted and then subjected to mass spectrometry.

The peptide fractions which are obtained in this way from the step of separation and purification are subjected to mass spectrometry (MS) to identify proteins in the sample. Various procedures and methods for performing MS measurements are known and many instruments therefor are commercially available, so that selection can be made as appropriate to use them. Additionally, in order to seek improvements in performance of separation and qualitative determination, analytical procedures have been developed which combine gas chromatography (GC) or liquid chromatography (LC) with MS (GC/MS, LC/MS, LC/MS/MS, and the like), and many instruments for those procedures are commercially available. In particular, LC/MS is suitable for analysis of proteins and peptides as in the present invention. In the specification, not only mere MS, but also configurations incorporating MS, such as GC/MS, LC/MS, and LC/MS/MS are referred to as mass spectrometry (MS). Ionization methods in MS usually use electrospray ionization (ESI), atmospheric pressure chemical ionization (APCI), matrix-assisted laser desorption ionization (MALDI) methods, and methods for analyzing ionized fragments include, for example, ion trap, time of flight, quadrupole, Fourier transform, and other methods, and thus selection can be made as appropriate to use them.

As described above, the method of the present invention is a method in which the tag is cleaved in a lump prior to the separation and purification of the labeled peptides, and therefore does not require applying, as in the conventional procedures, each of the peptide fractions which are obtained in the step of separation and purification to tag cleavage treatment, and thus can save time and labor. In addition, the method of the present invention is a method suitable for the identification/quantification of a wide variety of small-amount proteins in samples. Accordingly, the method of the present invention is suitable for a high throughput analysis of a wide variety of small-amount proteins in samples. Therefore, the present invention, in a further embodiment, provides a system for performing an analysis of differential expression of small-amount proteins in samples, the system characterized by employing the method of the present invention as described above. The system of the present invention is suitable, for example, for an analysis of differential expression, preferably a high throughput analysis, of proteins in serum samples of mammalian animals, in particular, of humans.

The present invention will now be described specifically and in detail by way of examples, which are intended to be only illustrative of the present invention and not to be limiting of the scope of the present invention.

### EXAMPLES

1) Removal of major proteins in serum by an Agilent antibody column:

A serum fraction which was obtained by employing an Agilent antibody column (for the removal of albumin, IgG, α1-antitrypsin, IgA, transferin, and haptoglobin, 10×100 mm) to remove the six major serum proteins described above was used for analysis. Accordingly, 200 μl of human serum (Rockland Immunochemicals, Inc.) was centrifuged at 15,000 rpm, diluted 5 times in Agilent Binding Buffer A, filtered through a 0.22 μm filter, and loaded onto the above-described antibody column to collect the flow-through fraction in which the six major proteins described above had been removed on the above-described antibody column. The flow-through fraction was concentrated and buffer changed on a Centriprep centrifugation filter unit (YM-3, Millipore) to 50 mM Tris/HCI, 0.1% SDS (pH 8.5), followed by determining the protein concentration by Lowry method.

2) cICAT reaction of human normal serum:

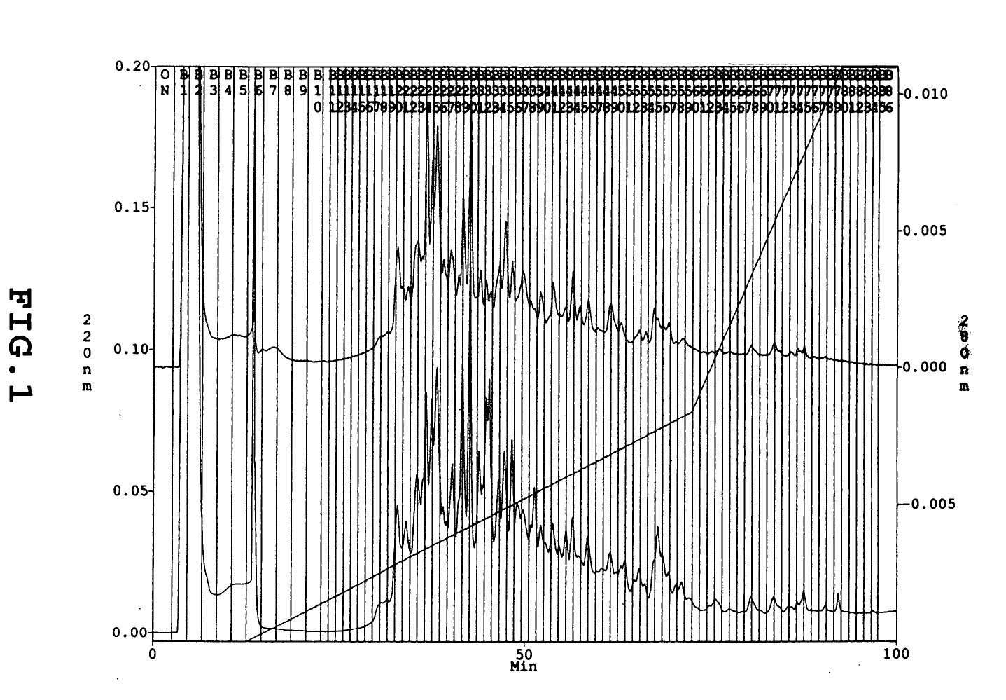

The serum protein faction in which the six major serum protein described above had been removed (a final concentration of 1 mg/ml) was solubilized in 50 mM Tris/HCl, 0.1% SDS (pH8.5), reduced with TCEP (a final concentration of 1 mM; at 95° C. for 10 min.), and then reacted with 2.2 mM of a Cleavable ICAT reagent (Applied Biosystem (ABI), ¹³C (H chain) or ¹²C (L chain) label) at 37° C. for 2 hours. An unreacted reagent was quenched with 1.0 mM TCEP, and the H-chain and L-chain samples were mixed at an equal amount and subjected to digestion with trypsin (Promega, TPCK treated) at 37° C. for 16 hours. The resultant digestion was loaded onto an SCX column (poly-LC-sulphoethyl A column (4.6×100 mm)) using a Vision Workstation system (ABI). After adsorption and washing in 10 mM KH₂PO₄, pH 2.8, 25% CH₃CN (SCX binding buffer), elution was carried out with the SCX binding buffer plus 0.5 M KCl (SCX elution buffer). The eluted fraction was applied to a large avidin-column (6.2×66.5 mm), the flow-through portion was washed, and the adsorbed cICAT-reagent-reacted peptides were eluted with 30% CH₃CN/0.4% TFA (using the Vision Workstation System) . The eluted fraction was dried and then reacted with 95% TFA (containing 5% scavenger) at 37° C. for 2 hours to cleave the biotin segment to obtain the ICAT-labeled peptides (H and L chains). The reaction mixture containing these peptides was subjected to dryness under reduced pressure, and then dissolved in the SCX binding buffer. The peptide solution was applied again to an SCX column, which was washed thoroughly with the SCX binding buffer to remove fractions of the tag and others. After that, the SCX binding buffer plus KCl (gradient of 0 to 0.5 M) was used to fractionate the peptides (50 fractions) (FIG. 1). Each of these fractions was desalted on a C18 trap column and subjected to dryness under reduced pressure.

3) Separation and purification of cICAT-peptides by nano-LC:

The ICAT-labeled peptides which were fractionated and desalted by SCX were re-dissolved in 0.1% TFA-2% CH₃CN and analyzed on nano-LC (LC-Packings)/Q-Star XL (ABI, ESI-Q/TOF, hereinafter referred to as “Q-Star”) and on nano-LC/Probot (LC-Packings)/ABI-4700 Proteomics Analyzer (ABI, MALDI-TOF/TOF, hereinafter referred to as “ABI-4700”) (column: PepMap™ C18 100, 3 μm, 100 angstroms, 75 μm (i.d.)×150 mm (LC-Packings), mobile phase for Q-Star: a linear gradient of A: 5% CH₃CN/0.1% HCOOH and B: 95% CH₃CN/0.1% HCOOH, mobile phase for ABI-4700: a linear gradient of A: 5% CH₃CN/0.1% TFA and B: 95% CH₃CN/0.1% TFA). Each mass spectrometry was performed as follows.

4) Measurements on Q-Star (ESI-Q/TOF):

A BSA digestion (50 fmol) was used to adjust the nano-LC. After confirming that a predetermined sequence coverage (a degree of about 40%) was achieved, measurements of samples were made according to the routine procedure. Measurements were made in an automatic measurement mode (IDA mode) in which one cycle is of a total of 7 seconds: MS for 1 second, 1st MS/MS for 3 seconds, and 2nd MS/MS for 3 seconds.

5) Measurements on ABI-4700 (MALDI-TOF/TOF):

A sample was separated on the nano-LC/Probot system and spotted with a matrix (CHCA, 875 ng/well). A sample plate was inserted into the apparatus, and then the laser intensity was determined on an MS reflector mode for the measurement of calibrants (Des-[Argl]-bradykinin (M+H)⁺=904.468; angiotensin I (M+H)⁺=1296.685; ACTH (1-17) (M+H)⁺=2093.087; ACTH (18-39) (M+H)⁺=2465.199; ACTH (7-38) (M+H)⁺=3657.929). Subsequently, some of the spots where the sample was applied were randomly selected and the laser intensity was determined for MS and MS/MS measurements. After that, a method for automatic measurements was prepared and MS-MS/MS sequential measurements were made (MS accumulations: 1250, MS/MS accumulations: 2000).

6) Results of the analysis of human serum protein by the cICAT method:

The date obtained by the above-described analytical instruments for mass spectrometry were analyzed employing a combined data identification system (HiSpec) using RefSeq as the DB to be searched, and peptides and proteins were identified and the H and L chains were comparatively quantified. Since the H-chain and L-chain labels were allowed to be reacted at an equal amount (as described above), the ratio of H-chain labeling and L-chain labeling would theoretically be 1. Results are shown in Table 1, which ranks identified protein in decreasing order of total score (Rank, Q-Star or ABI-4700) and summarizes their generic names (Description), GI numbers, molecular weights (Mass), score values of the H and L chains, ratios of the H/L chains (Ratio, comparative quantification value), the number of Cys residues (Total cys), the number of trypsin-digestion fragments actually identified of the H- and L-chain labeling reactions (NRPepCnt (H, L)), and sequence coverages (Protein Coverage (H, L)).

| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | H factor 1 (complement); H factor-1 | [symbol] | [symbol] | 139125.4 | [symbol] | [symbol] | 0.92 | 0.97 | [symbol] | [symbol] | 1563.5 | 933.8 | [symbol] | [symbol] | 30 | 25 |
| | | (complement); complement factor H; | | | | | | | | | | | | | | | |
| | | factor H-like 1; H factor 2 (complement) | | | | | | | | | | | | | | | |
| [symbol] | [symbol] | plastinogen | [symbol] | [symbol] | 90689 | [symbol] | [symbol] | 0.95 | 0.96 | [symbol] | [symbol] | 1159.5 | 882.6 | [symbol] | [symbol] | 34 | 33 |
| [symbol] | [symbol] | coagulation factor II precursor; | [symbol] | [symbol] | 70036.9 | [symbol] | [symbol] | 0.98 | 0.98 | [symbol] | [symbol] | 959.7 | 707.5 | [symbol] | [symbol] | 32 | 35 |
| [symbol] | [symbol] | alpha 2 macroglobulin precursor | [symbol] | [symbol] | 163278 | [symbol] | [symbol] | 0.92 | 0.95 | [symbol] | [symbol] | 1222.9 | 884.8 | [symbol] | [symbol] | 15 | 15 |
| [symbol] | [symbol] | complement component 3 precursor | [symbol] | [symbol] | 187164.1 | [symbol] | [symbol] | 0.92 | 0.88 | [symbol] | [symbol] | 993.4 | 832.1 | [symbol] | [symbol] | 11 | 10 |
| | | a cylation-stimulating protein cleavage product | | | | | | | | | | | | | | | |
| [symbol] | [symbol] | transferrin; PRO2086 protein | [symbol] | [symbol] | 77049.9 | [symbol] | [symbol] | 0.83 | 0.92 | [symbol] | [symbol] | 791.9 | 595 | [symbol] | [symbol] | 25 | 28 |
| [symbol] | [symbol] | kininogen 1; alpha-2-thick proteinase inhibitor; | [symbol] | [symbol] | 47883.2 | [symbol] | [symbol] | 0.96 | 0.94 | [symbol] | [symbol] | 626.8 | 453.3 | [symbol] | [symbol] | 29 | 27 |
| | | bradykinin | | | | | | | | | | | | | | | |
| [symbol] | [symbol] | [symbol] amin precursor; alpha-albumin | [symbol] | [symbol] | 69069.1 | [symbol] | [symbol] | 0.95 | 0.99 | [symbol] | [symbol] | 638.7 | 432.6 | [symbol] | [symbol] | 21 | 21 |
| [symbol] | [symbol] | vitamin D-binding protein precursor; | [symbol] | [symbol] | 52917.5 | [symbol] | [symbol] | 0.94 | 0.92 | [symbol] | [symbol] | 842.5 | 412.2 | [symbol] | [symbol] | 29 | 25 |
| | | vitamin D-binding alpha-globulin | | | | | | | | | | | | | | | |
| [symbol] | [symbol] | cerulaplasmin (ferroxidase); | [symbol] | [symbol] | 122205.2 | [symbol] | [symbol] | 0.96 | 0.92 | [symbol] | [symbol] | 397.6 | 395.8 | [symbol] | [symbol] | 9 | 12 |
| [symbol] | [symbol] | alpha-1-microglobulin/bikunin precursor; | [symbol] | [symbol] | 36999.5 | [symbol] | [symbol] | 0.93 | 0.88 | [symbol] | [symbol] | 493.1 | 363.3 | [symbol] | [symbol] | 30 | 33 |
| | | Alpha-1-microglobulin/bikunin precursor | | | | | | | | | | | | | | | |
| | | (inter-alpha-trypsin inhibitor, tight chain; protein | | | | | | | | | | | | | | | |
| | | HC); Alpha-1- | | | | | | | | | | | | | | | |
| | | microglobulin/bikunin precursor; inter- | | | | | | | | | | | | | | | |
| [symbol] | [symbol] | complement component 4A | [symbol] | [symbol] | 192336 | [symbol] | [symbol] | | 0.83 | [symbol] | [symbol] | | 355.4 | [symbol] | [symbol] | | 6 |
| | | preproprotein; acidic C4; Rodgers form of C4; | | | | | | | | | | | | | | | |
| | | C4A anaphytatoxin | | | | | | | | | | | | | | | |
| [symbol] | [symbol] | complement component 4B proprotein | [symbol] | [symbol] | 192797.5 | [symbol] | [symbol] | 0.92 | 0.83 | [symbol] | [symbol] | 490.3 | | [symbol] | [symbol] | 6 | |
| [symbol] | [symbol] | beta-2-glycoprotein I precursor | [symbol] | [symbol] | 36312.2 | [symbol] | [symbol] | 0.95 | 0.87 | [symbol] | [symbol] | 588.8 | 222.5 | [symbol] | [symbol] | 39 | 40 |
| [symbol] | [symbol] | I factor (complement) | [symbol] | [symbol] | 65788.3 | [symbol] | [symbol] | 0.92 | 0.9 | [symbol] | [symbol] | 507.1 | 319.8 | [symbol] | [symbol] | 28 | 17 |
| [symbol] | [symbol] | hemopexin | [symbol] | [symbol] | 51676.4 | [symbol] | [symbol] | 0.95 | 1.01 | [symbol] | [symbol] | 500.2 | 305.5 | [symbol] | [symbol] | 23 | 19 |
| [symbol] | [symbol] | complement factor B preproprotein; C3 | [symbol] | [symbol] | 85504.8 | [symbol] | [symbol] | 0.92 | 0.92 | [symbol] | [symbol] | 343.4 | 301.8 | [symbol] | [symbol] | 10 | 13 |
| | | proactivator; C3 proaccelerator; glycine-rich | | | | | | | | | | | | | | | |
| | | beta-glycoprotein; C3/C5 convartase | | | | | | | | | | | | | | | |
| [symbol] | [symbol] | complement component 7 precursor | [symbol] | [symbol] | 93518.2 | [symbol] | [symbol] | 0.93 | 0.91 | [symbol] | [symbol] | 650.7 | 297.6 | [symbol] | [symbol] | 18 | 15 |
| [symbol] | [symbol] | coagulation factor XIII B subunit precursor; | [symbol] | [symbol] | 75491.6 | [symbol] | [symbol] | 0.82 | 0.92 | [symbol] | [symbol] | 303.2 | 296.5 | [symbol] | [symbol] | 13 | 14 |
| | | TGase | | | | | | | | | | | | | | | |
| [symbol] | [symbol] | fibronectin 1 isoform 3 preproprotein; | [symbol] | [symbol] | 259225.9 | [symbol] | [symbol] | 1.05 | 1.11 | [symbol] | [symbol] | 346.9 | 205.9 | [symbol] | [symbol] | 4 | 2 |
| | | cold-insoluble globulin; migration-stimulating | | | | | | | | | | | | | | | |
| | | factor | | | | | | | | | | | | | | | |
| [symbol] | [symbol] | complement component 1. r | [symbol] | [symbol] | 80199.7 | [symbol] | [symbol] | 1.04 | 0.91 | [symbol] | [symbol] | 454.5 | 293.9 | [symbol] | [symbol] | 15 | 16 |
| [symbol] | [symbol] | complement component 4 binding | [symbol] | [symbol] | 87033.2 | [symbol] | [symbol] | 0.9 | 0.91 | [symbol] | [symbol] | 441.5 | 251.1 | [symbol] | [symbol] | 19 | 18 |
| | | protein, alpha; Complement component 4-binding | | | | | | | | | | | | | | | |
| | | protein, alpha polypeptide; complement | | | | | | | | | | | | | | | |
| | | component 4-binding | | | | | | | | | | | | | | | |
| [symbol] | [symbol] | alpha-2-HS-glycoprotein; Alpha-2HS- | [symbol] | [symbol] | 39324.7 | [symbol] | [symbol] | 1.01 | 0.93 | [symbol] | [symbol] | 398.6 | 241 | [symbol] | [symbol] | 20 | 30 |
| | | glycoprotein | | | | | | | | | | | | | | | |
| [symbol] | [symbol] | plasma [symbol] B1 precursor [symbol] | [symbol] | [symbol] | 71369.7 | [symbol] | [symbol] | 0.91 | 1.08 | [symbol] | [symbol] | 531 | 248.4 | [symbol] | [symbol] | 22 | 18 |
| | | plasma; [symbol] 3, plasma; [symbol] B | | | | | | | | | | | | | | | |
| | | plasma; Fleicher factor | | | | | | | | | | | | | | | |
| [symbol] | [symbol] | peptidoglycan recognition protein L precursor | [symbol] | [symbol] | 67970.3 | [symbol] | [symbol] | 0.97 | 0.98 | [symbol] | [symbol] | 324.9 | 228 | [symbol] | [symbol] | 12 | 11 |
| [symbol] | [symbol] | albumin precursor; PRO0883 protein | [symbol] | [symbol] | 69366.7 | [symbol] | [symbol] | 0.9 | 0.91 | [symbol] | [symbol] | 491.9 | 220.7 | [symbol] | [symbol] | 18 | [symbol] |
| [symbol] | [symbol] | Complement component B precursor | [symbol] | [symbol] | 104844.1 | [symbol] | [symbol] | 1.01 | 0.99 | [symbol] | [symbol] | 300.3 | 171.9 | [symbol] | [symbol] | 10 | [symbol] |
| [symbol] | [symbol] | apolipoprotein D precursor | [symbol] | [symbol] | 21275.6 | [symbol] | [symbol] | 1 | 1.02 | [symbol] | [symbol] | 175.2 | 139.9 | [symbol] | [symbol] | 13 | 13 |
| [symbol] | [symbol] | properdin P factor, complement | [symbol] | [symbol] | 51278.4 | [symbol] | [symbol] | 0.99 | 1.05 | [symbol] | [symbol] | 151.2 | 116.1 | [symbol] | [symbol] | 10 | 10 |
| [symbol] | [symbol] | complement component B. alpha | [symbol] | [symbol] | 86183.2 | [symbol] | [symbol] | 0.83 | 1 | [symbol] | [symbol] | 340.9 | 168.1 | [symbol] | [symbol] | 19 | 10 |
| | | polypeptide precursor | | | | | | | | | | | | | | | |
| [symbol] | [symbol] | complement component 1, 3 | [symbol] | [symbol] | 76884.4 | [symbol] | [symbol] | 1.02 | 0.88 | [symbol] | [symbol] | 243.2 | 183.2 | [symbol] | [symbol] | 8 | 6 |
| [symbol] | [symbol] | [symbol] (plasminogen binding | [symbol] | [symbol] | 22586.8 | [symbol] | [symbol] | 0.78 | 1 | [symbol] | [symbol] | 159.4 | 180.9 | [symbol] | [symbol] | 17 | 17 |
| | | protein); [symbol] (plasminogen- | | | | | | | | | | | | | | | |
| [symbol] | [symbol] | [symbol] precursor; serum spreading | [symbol] | [symbol] | 54335.7 | [symbol] | [symbol] | 0.95 | 0.96 | [symbol] | [symbol] | 236.2 | 140.2 | [symbol] | [symbol] | 7 | 7 |
| | | factor; somatomedin B, complement S- | | | | | | | | | | | | | | | |
| | | protein; | | | | | | | | | | | | | | | |
| [symbol] | [symbol] | apolipoprotein B precursor, apoB-100; | [symbol] | [symbol] | 515862.7 | [symbol] | [symbol] | 1.02 | 0.96 | [symbol] | [symbol] | 135.5 | 155.6 | [symbol] | [symbol] | 1 | 1 |
| | | apoB-16 | | | | | | | | | | | | | | | |
| [symbol] | [symbol] | apolipoprotein M; NG20-like protein | [symbol] | [symbol] | 21253.3 | [symbol] | [symbol] | 0.91 | 0.96 | [symbol] | [symbol] | 98.8 | 147.8 | [symbol] | [symbol] | 12 | 25 |
| [symbol] | [symbol] | attractin isoform 1; attractin-2; mahogany | [symbol] | [symbol] | 158836.9 | [symbol] | [symbol] | 0.69 | 1.2 | [symbol] | [symbol] | 226.2 | 110.4 | [symbol] | [symbol] | 8 | 4 |
| | | protein | | | | | | | | | | | | | | | |
| [symbol] | [symbol] | coagulation factor XII precursor | [symbol] | [symbol] | 67618.1 | [symbol] | [symbol] | 0.83 | 1.02 | [symbol] | [symbol] | 243.3 | 132 | [symbol] | [symbol] | 9 | 7 |
| | | Hageman factor | | | | | | | | | | | | | | | |
| [symbol] | [symbol] | complement component 8, beta | [symbol] | [symbol] | 66947.7 | [symbol] | [symbol] | 0.99 | 0.94 | [symbol] | [symbol] | 80.5 | 127.1 | [symbol] | [symbol] | 11 | 10 |
| [symbol] | [symbol] | alpha-2-glycoprotein 1, zinc; Alpha-2- | [symbol] | [symbol] | 34258.7 | [symbol] | [symbol] | 1.05 | 0.93 | [symbol] | [symbol] | 108.3 | 121.2 | [symbol] | [symbol] | 7 | 7 |
| | | glycoprotein, zinc | | | | | | | | | | | | | | | |
| [symbol] | [symbol] | serine (or cysteine) proteinase inhibitor, | [symbol] | [symbol] | 52602.4 | [symbol] | [symbol] | 0.95 | 1.1 | [symbol] | [symbol] | 165.6 | 115 | [symbol] | [symbol] | 7 | 7 |
| | | clade C (antithrombin), member 1; | | | | | | | | | | | | | | | |
| | | antithrombin III | | | | | | | | | | | | | | | |
| [symbol] | [symbol] | haptoglobin | [symbol] | [symbol] | 45205.3 | [symbol] | [symbol] | 0.77 | 0.89 | [symbol] | [symbol] | 252.2 | 67.7 | [symbol] | [symbol] | 19 | 9 |
| [symbol] | [symbol] | alpha 1B-glycoprotein | [symbol] | [symbol] | 54253.5 | [symbol] | [symbol] | 0.94 | 0.91 | [symbol] | [symbol] | 240.7 | 111.4 | [symbol] | [symbol] | 8 | 8 |
| [symbol] | [symbol] | histidine-rich glycoprotein precursor; | [symbol] | [symbol] | 59675.3 | [symbol] | [symbol] | 0.95 | 0.92 | [symbol] | [symbol] | 258.7 | 105.2 | [symbol] | [symbol] | 10 | 8 |
| | | histidine-proline rich glycoprotein; | | | | | | | | | | | | | | | |
| | | thrombophilia due to elevated HRG, | | | | | | | | | | | | | | | |
| [symbol] | [symbol] | complement component 4 binding | [symbol] | [symbol] | 28357.4 | [symbol] | [symbol] | 0.96 | 1.03 | [symbol] | [symbol] | 92.8 | 88.3 | [symbol] | [symbol] | 9 | 15 |
| | | protein, beta; complement component 4- | | | | | | | | | | | | | | | |
| | | binding protein, beta polypeptide; | | | | | | | | | | | | | | | |
| | | complement component 4-binding | | | | | | | | | | | | | | | |
| [symbol] | [symbol] | immunoglobulin J chain | [symbol] | [symbol] | 18098.6 | [symbol] | [symbol] | 1.05 | 0.98 | [symbol] | [symbol] | 104.2 | 23 | [symbol] | [symbol] | 15 | 6 |
| [symbol] | [symbol] | [symbol] 1 precursor; [symbol] - | [symbol] | [symbol] | 23511.6 | [symbol] | [symbol] | 0.98 | 0.9 | [symbol] | [symbol] | 156.5 | 93.1 | [symbol] | [symbol] | 10 | 10 |
| | | 1 (alpha-1-acid glycoprotein-1); alpha-1- | | | | | | | | | | | | | | | |
| | | acid glycoprotein 1 | | | | | | | | | | | | | | | |
| [symbol] | [symbol] | complement component 2 precursor; | [symbol] | [symbol] | 83287.8 | [symbol] | [symbol] | 1.07 | 1.25 | [symbol] | [symbol] | 64 | 28.2 | [symbol] | [symbol] | 4 | 3 |
| | | C3/C5 convertase | | | | | | | | | | | | | | | |
| [symbol] | [symbol] | alpha-2-plasmin inhibitor, alpha-2- | [symbol] | [symbol] | 54595.8 | [symbol] | [symbol] | 0.94 | 0.83 | [symbol] | [symbol] | 87 | 97 | [symbol] | [symbol] | 6 | 6 |
| | | antiplasmin | | | | | | | | | | | | | | | |
| [symbol] | [symbol] | orosomucold 2; alpha-1-acid | [symbol] | [symbol] | 23602.6 | [symbol] | [symbol] | 0.97 | 0.81 | [symbol] | [symbol] | 89.5 | 82.7 | [symbol] | [symbol] | 12 | 12 |
| | | glycoprotein, type 2 | | | | | | | | | | | | | | | |
| [symbol] | [symbol] | protein S (alpha); Protein S, alpha | [symbol] | [symbol] | 75072.5 | [symbol] | [symbol] | 0.94 | 1 | [symbol] | [symbol] | 164.8 | 95.6 | [symbol] | [symbol] | 8 | 3 |
| [symbol] | [symbol] | clustarin isoform 1; complement- | [symbol] | [symbol] | 57832.6 | [symbol] | [symbol] | 2.29 | 0.94 | [symbol] | [symbol] | 145.6 | 94.9 | [symbol] | [symbol] | 8 | 4 |
| | | associated protein SP-40 | | | | | | | | | | | | | | | |
| [symbol] | [symbol] | complement component 9 | [symbol] | [symbol] | 63173.4 | [symbol] | [symbol] | 0.98 | 0.89 | [symbol] | [symbol] | 160.7 | 92.9 | [symbol] | [symbol] | 6 | 7 |
| [symbol] | [symbol] | inter-alpha (globulin) inhibitor H1; Inter- | [symbol] | [symbol] | 101402.2 | [symbol] | [symbol] | 0.94 | 0.92 | [symbol] | [symbol] | 31.5 | 87.4 | [symbol] | [symbol] | 2 | 2 |
| | | alpha (globulin) inhibitor, H1 polypeptide | | | | | | | | | | | | | | | |
| [symbol] | [symbol] | mannan-binding lectin serine protease I | [symbol] | [symbol] | 79246.7 | [symbol] | [symbol] | 1 | 0.95 | [symbol] | [symbol] | 130 | 30.3 | [symbol] | [symbol] | 6 | 2 |
| | | Isoform 1, precursor, protease, serine, 5 | | | | | | | | | | | | | | | |
| | | (mannose-binding protein-associated); | | | | | | | | | | | | | | | |
| | | manan-binding lectin serine protease-1; | | | | | | | | | | | | | | | |
| | | Re-reactive factor serine protease p100 | | | | | | | | | | | | | | | |
| [symbol] | [symbol] | complement component 8, gamma | [symbol] | [symbol] | 22219.4 | [symbol] | [symbol] | 1.11 | 1.01 | [symbol] | [symbol] | 29.3 | 40.3 | [symbol] | [symbol] | 8 | 8 |
| | | polypeptide | | | | | | | | | | | | | | | |
| [symbol] | [symbol] | complement component 5 | [symbol] | [symbol] | 188305.3 | [symbol] | [symbol] | 1.01 | 0.97 | [symbol] | [symbol] | 228.6 | 80.6 | [symbol] | [symbol] | 3 | 2 |
| [symbol] | [symbol] | bintinidase precursor | [symbol] | [symbol] | 61132.9 | [symbol] | [symbol] | 0.94 | 0.9 | [symbol] | [symbol] | 145.5 | 42.5 | [symbol] | [symbol] | 7 | 3 |
| [symbol] | [symbol] | PREDICTED: [symbol] to | [symbol] | [symbol] | 85603.6 | [symbol] | [symbol] | 1.02 | 1.01 | [symbol] | [symbol] | 40.7 | 78.6 | [symbol] | [symbol] | 2 | 7 |
| | | Carboxypeptidase N 83 KDa chain | | | | | | | | | | | | | | | |
| | | (Carboxypeptidase N regulatory subunit) | | | | | | | | | | | | | | | |
| [symbol] | [symbol] | apolipoprotein A-II precursor | [symbol] | [symbol] | 11175 | [symbol] | [symbol] | 1 | 0.75 | [symbol] | [symbol] | 114.1 | 61.7 | [symbol] | [symbol] | 20 | 20 |
| [symbol] | [symbol] | serine (or cysteine) proteinase inhibitor, | [symbol] | [symbol] | 47850.9 | [symbol] | [symbol] | 0.88 | 0.87 | [symbol] | [symbol] | 17.2 | 45.1 | [symbol] | [symbol] | 2 | 2 |
| | | clude A, member 3 precurseor, alpha-1- | | | | | | | | | | | | | | | |
| | | antichymotrypsin; antichymotrydain | | | | | | | | | | | | | | | |
| [symbol] | [symbol] | haplogtobin-reisted protein; Haptoglobin- | [symbol] | [symbol] | 39029.6 | [symbol] | [symbol] | | 0.86 | [symbol] | [symbol] | | 58.5 | [symbol] | [symbol] | | 10 |
| | | related locus | | | | | | | | | | | | | | | |
| [symbol] | [symbol] | coagulation factor V precursor; labile | [symbol] | [symbol] | 251719.4 | [symbol] | [symbol] | 0.82 | 0.87 | [symbol] | [symbol] | 56.6 | 5.9 | [symbol] | [symbol] | 1 | 1 |
| | | factor, factor V Laiden | | | | | | | | | | | | | | | |
| [symbol] | [symbol] | Insulin-like growth factor binding protein, | [symbol] | [symbol] | 66035 | [symbol] | [symbol] | 0.92 | 0.97 | [symbol] | [symbol] | 77.3 | 39.8 | [symbol] | [symbol] | 3 | 2 |
| | | acid labile submit; INSULIN-LIKE | | | | | | | | | | | | | | | |
| | | GROWTH FACTOR BINDING PROTEIN | | | | | | | | | | | | | | | |
| | | COMPLEX ACID LABLE CHAIN | | | | | | | | | | | | | | | |
| [symbol] | [symbol] | M factor (complement)-like 3; factor H- | [symbol] | [symbol] | 30650.7 | [symbol] | [symbol] | 1 | 1.08 | [symbol] | [symbol] | 108.7 | 40 | [symbol] | [symbol] | 11 | 7 |
| | | ralated gene 2 | | | | | | | | | | | | | | | |
| [symbol] | [symbol] | H factor (complement)-like 1 | [symbol] | [symbol] | 37881.8 | [symbol] | [symbol] | 0.91 | 0.95 | [symbol] | [symbol] | 143 | 61.5 | [symbol] | [symbol] | 12 | 8 |
| [symbol] | [symbol] | sex hormone-binding globulin; Sex | [symbol] | [symbol] | 43778.2 | [symbol] | [symbol] | 0.99 | 1.01 | [symbol] | [symbol] | 26 | 60.3 | [symbol] | [symbol] | 2 | 2 |
| | | hormone-binding globulin (androgen | | | | | | | | | | | | | | | |
| | | binding protein) | | | | | | | | | | | | | | | |
| [symbol] | [symbol] | keratin 1; Keratin-1; cytokeratin 1; hair | [symbol] | [symbol] | 68066.7 | [symbol] | [symbol] | N/A | 1.08 | [symbol] | [symbol] | 10.6 | 57.6 | [symbol] | [symbol] | 4 | 4 |
| | | alpha protein | | | | | | | | | | | | | | | |
| [symbol] | [symbol] | apolipoprotein E precursor; | [symbol] | [symbol] | 36154.1 | [symbol] | [symbol] | 1.08 | 1.01 | [symbol] | [symbol] | 37.5 | 57.3 | [symbol] | [symbol] | 3 | 3 |
| | | apolipoprotein E3 | | | | | | | | | | | | | | | |
| [symbol] | [symbol] | Insulin-like growth factor binding protein | [symbol] | [symbol] | 31660.2 | [symbol] | [symbol] | 1.02 | 1.1 | [symbol] | [symbol] | 33.4 | 54.4 | [symbol] | [symbol] | 6 | 12 |
| [symbol] | [symbol] | cysteine-rich secretory protein 3; specific | [symbol] | [symbol] | 27630.3 | [symbol] | [symbol] | 0.77 | 0.87 | [symbol] | [symbol] | 45 | 44.2 | [symbol] | [symbol] | 8 | 3 |
| | | granule protein (28 kDa); cysteine-rich | | | | | | | | | | | | | | | |
| | | secretory protein-3 | | | | | | | | | | | | | | | |
| [symbol] | [symbol] | HGF activator preproprotein | [symbol] | [symbol] | 70661.8 | [symbol] | [symbol] | 0.88 | 1.14 | [symbol] | [symbol] | 18.3 | 28.8 | [symbol] | [symbol] | 1 | 1 |
| [symbol] | [symbol] | gelsolin isoform a | [symbol] | [symbol] | 85697.6 | [symbol] | [symbol] | 0.81 | 0.9 | [symbol] | [symbol] | 105.9 | 38.4 | [symbol] | [symbol] | 1 | 1 |
| [symbol] | [symbol] | apolipoprotein F precursor | [symbol] | [symbol] | 36399.6 | [symbol] | [symbol] | 0.86 | 1.49 | [symbol] | [symbol] | 41 | | [symbol] | [symbol] | 3 | |
| [symbol] | [symbol] | CD5 antigen-like (scavenger receptor | [symbol] | [symbol] | 38087.8 | [symbol] | [symbol] | 1.02 | 1.03 | [symbol] | [symbol] | 140.5 | 46.7 | [symbol] | [symbol] | 14 | 6 |
| | | cysteine rich family); [symbol] ; apoptosis | | | | | | | | | | | | | | | |
| | | Inhibitor B | | | | | | | | | | | | | | | |
| [symbol] | [symbol] | hyaluronan binding protein 2; hyaluronic | [symbol] | [symbol] | 52671.7 | [symbol] | [symbol] | 1.04 | 0.94 | [symbol] | [symbol] | 145.7 | 36.6 | [symbol] | [symbol] | 6 | 2 |
| | | acid binding protein 2, hepatocyte growth | | | | | | | | | | | | | | | |
| | | factor activator-like protein; plasma | | | | | | | | | | | | | | | |
| | | hyaluronan binding protein; factor VII | | | | | | | | | | | | | | | |
| | | activating protein; hyaluronan-binding | | | | | | | | | | | | | | | |
| | | protein 2 | | | | | | | | | | | | | | | |
| [symbol] | [symbol] | protein C (inactivator of coagulation | [symbol] | [symbol] | 52071.3 | [symbol] | [symbol] | 1.2 | 1.02 | [symbol] | [symbol] | 59.5 | 45.7 | [symbol] | [symbol] | 5 | 9 |
| | | factore Va and VIIIa) | | | | | | | | | | | | | | | |
| [symbol] | [symbol] | feluin B; feluin-like protein | [symbol] | [symbol] | 42094 | [symbol] | [symbol] | 0.96 | 0.75 | [symbol] | [symbol] | 84.3 | 44.6 | [symbol] | [symbol] | 2 | 2 |
| [symbol] | [symbol] | extracellular matrix protein 1 isoform 1 | [symbol] | [symbol] | 60704.1 | [symbol] | [symbol] | 1.1 | 0.89 | [symbol] | [symbol] | 21.7 | 43.1 | [symbol] | [symbol] | 1 | 3 |
| | | precursor; secretory component p85 | | | | | | | | | | | | | | | |
| | | precursor; secretory component p85 | | | | | | | | | | | | | | | |
| [symbol] | [symbol] | lumican | [symbol] | [symbol] | 38429 | [symbol] | [symbol] | 1 | 1 | [symbol] | [symbol] | 59.1 | 22.2 | [symbol] | [symbol] | 4 | 4 |
| [symbol] | [symbol] | protein Z, vitamin K-dependent plasma | [symbol] | [symbol] | 44743.9 | [symbol] | [symbol] | 0.97 | 0.85 | [symbol] | [symbol] | 85.3 | 41.2 | [symbol] | [symbol] | 7 | 3 |
| | | glycoprotein | | | | | | | | | | | | | | | |
| [symbol] | [symbol] | CD44 antigen isoform 1 precursor, cell | [symbol] | [symbol] | 81537.6 | [symbol] | [symbol] | | 0.94 | [symbol] | [symbol] | | 40.7 | [symbol] | [symbol] | | 1 |
| | | surface glycoprotein CD44; Lutheran | | | | | | | | | | | | | | | |
| | | inhibitor, dominant; homing function and | | | | | | | | | | | | | | | |
| | | Indian blood group system; monoclonal | | | | | | | | | | | | | | | |
| | | antibody A3D8; antigen gp90 homing | | | | | | | | | | | | | | | |
| | | receptor, CDW44 antigen; phagocylic | | | | | | | | | | | | | | | |
| | | glycoprotein I; extracellular | | | | | | | | | | | | | | | |
| [symbol] | [symbol] | serine (or cysteine) proteinase inhibitor, | [symbol] | [symbol] | 46312.2 | [symbol] | [symbol] | 2.11 | 0.91 | [symbol] | [symbol] | 26.9 | | [symbol] | [symbol] | 2 | |
| | | clade F (alpha-2 antiplasmin, pigment | | | | | | | | | | | | | | | |
| | | epithelium derived factor, member 1; | | | | | | | | | | | | | | | |
| | | pigment epithelium-derived factor | | | | | | | | | | | | | | | |
| [symbol] | [symbol] | platelet glycoprotein Ib alpha polypeptide | [symbol] | [symbol] | 68955.3 | [symbol] | [symbol] | 1.13 | 0.95 | [symbol] | [symbol] | 32.6 | 39.5 | [symbol] | [symbol] | 1 | 5 |
| | | precursor, platelet membrane | | | | | | | | | | | | | | | |
| | | glycoprotein 1b-alpha subunit1 | | | | | | | | | | | | | | | |
| [symbol] | [symbol] | macrophage stimulating 1 (hepatocyte | [symbol] | [symbol] | 80319.9 | [symbol] | [symbol] | 0.99 | 1.01 | [symbol] | [symbol] | 27.8 | 39.4 | [symbol] | [symbol] | 0 | 1 |
| | | growth factor-like) | | | | | | | | | | | | | | | |
| [symbol] | [symbol] | complement component 1, q | [symbol] | [symbol] | 26016.8 | [symbol] | [symbol] | 0.85 | 0.91 | [symbol] | [symbol] | | | [symbol] | [symbol] | | |
| | | subcomponent, alpha polypeptide | | | | | | | | | | | | | | | |
| | | precursor, complement component C1q. | | | | | | | | | | | | | | | |
| [symbol] | [symbol] | programmed cell death B isoform 1; | [symbol] | [symbol] | 66900.5 | [symbol] | [symbol] | | N/A | [symbol] | [symbol] | | 38 | [symbol] | [symbol] | | 1 |
| | | apoptosis-inducing factor | | | | | | | | | | | | | | | |
| [symbol] | [symbol] | plasma glutathione peroxidase 3 | [symbol] | [symbol] | 25402.3 | [symbol] | [symbol] | 1 | 0.9 | [symbol] | [symbol] | 58.2 | | [symbol] | [symbol] | 6 | |
| [symbol] | [symbol] | plasma carboxypeptidase B2 isoform a | [symbol] | [symbol] | 48442.2 | [symbol] | [symbol] | 0.93 | 0.84 | [symbol] | [symbol] | 101.4 | 37.6 | [symbol] | [symbol] | 8 | 3 |
| | | praproprotein; carboxypeptidase U; | | | | | | | | | | | | | | | |
| | | thrombin-activatable fibrinolysis inhibitor; | | | | | | | | | | | | | | | |
| | | carboxypeptidase B-like protein; | | | | | | | | | | | | | | | |
| | | thrombin-activable fibrinolysis inhibitor | | | | | | | | | | | | | | | |
| [symbol] | [symbol] | complement component 1, q | [symbol] | [symbol] | 25703.7 | [symbol] | [symbol] | 1.03 | 0.68 | [symbol] | [symbol] | 11.9 | 37.5 | [symbol] | [symbol] | 3 | 3 |
| | | subcomponent, beta polypeptide | | | | | | | | | | | | | | | |
| | | precursor; complement component C1q. | | | | | | | | | | | | | | | |
| [symbol] | [symbol] | heparin cofactor II | [symbol] | [symbol] | 57098.6 | [symbol] | [symbol] | | 1.24 | [symbol] | [symbol] | | | [symbol] | [symbol] | | |
| [symbol] | [symbol] | coagulation factor XIII A1 subunit | [symbol] | [symbol] | 83233.3 | [symbol] | [symbol] | | 0.81 | [symbol] | [symbol] | | | [symbol] | [symbol] | | |
| | | precursor, Coagulation factor XIII, A | | | | | | | | | | | | | | | |
| | | polypeptide; TGase | | | | | | | | | | | | | | | |
| [symbol] | [symbol] | complement component 1 inhibitor, | [symbol] | [symbol] | 55153.2 | [symbol] | [symbol] | 0.93 | 0.91 | [symbol] | [symbol] | 60.4 | 38.4 | [symbol] | [symbol] | 2 | 2 |
| | | precursor | | | | | | | | | | | | | | | |
| [symbol] | [symbol] | serine ( [symbol] cysteine) proteinase inhibitor, | [symbol] | [symbol] | 50707 | [symbol] | [symbol] | 0.8 | N/A | [symbol] | [symbol] | 25.4 | | [symbol] | [symbol] | 2 | |
| | | clade A (alpha-1 antiproteinase, | | | | | | | | | | | | | | | |
| | | [symbol] ), member 10; protein Z- | | | | | | | | | | | | | | | |
| | | dependent protease inhibitor precursor; | | | | | | | | | | | | | | | |
| | | protein Z-dependent protease | | | | | | | | | | | | | | | |
| | | inhibitor precursor | | | | | | | | | | | | | | | |
| [symbol] | [symbol] | cultin 1 | [symbol] | [symbol] | 89678.5 | [symbol] | [symbol] | 1.02 | 0.97 | [symbol] | [symbol] | | 27.6 | [symbol] | [symbol] | | 1 |
| [symbol] | [symbol] | coagulation factor X precursor | [symbol] | [symbol] | 54731.7 | [symbol] | [symbol] | 1.02 | 0.89 | [symbol] | [symbol] | 95.9 | 10.2 | [symbol] | [symbol] | 7 | 0 |
| | | prothrombinase; factor Xa | | | | | | | | | | | | | | | |
| [symbol] | [symbol] | carboxypeptidase N, polypeptide 1, 50 kD | [symbol] | [symbol] | 52286.2 | [symbol] | [symbol] | 1.02 | 1.01 | [symbol] | [symbol] | 18.5 | | [symbol] | [symbol] | 2 | |
| | | precursor | | | | | | | | | | | | | | | |
| [symbol] | [symbol] | inter-alpha (globulin) inhibitor H2; inter- | [symbol] | [symbol] | 105713.9 | [symbol] | [symbol] | 0.91 | 0.31 | [symbol] | [symbol] | 15.8 | 31.3 | [symbol] | [symbol] | 0 | 2 |
| | | alpha (globulin) inhibitor, H2 polypeptide | | | | | | | | | | | | | | | |
| [symbol] | [symbol] | coagulation factor IX; Coagulation factor | [symbol] | [symbol] | 51778.4 | [symbol] | [symbol] | 1.05 | 1.82 | [symbol] | [symbol] | 22.5 | | [symbol] | [symbol] | 2 | |
| | | IX (plasma thromboplastic component); | | | | | | | | | | | | | | | |
| | | Factor 9; Factor IX; Christmas factor | | | | | | | | | | | | | | | |
| [symbol] | [symbol] | hypothetical protein DKFZp434F1726 | [symbol] | [symbol] | 213146 | [symbol] | [symbol] | 2.48 | 0.85 | [symbol] | [symbol] | 10.4 | | [symbol] | [symbol] | 0 | |
| | | isoform 1 | | | | | | | | | | | | | | | |
| [symbol] | [symbol] | SMC6 protein | [symbol] | [symbol] | 126325.6 | [symbol] | [symbol] | 0.93 | 0.94 | [symbol] | [symbol] | 26.6 | | [symbol] | [symbol] | 1 | |
| [symbol] | [symbol] | ring finger protein 130; [symbol] protein; | [symbol] | [symbol] | 46404.9 | [symbol] | [symbol] | 0.93 | 4.27 | [symbol] | [symbol] | 16.6 | 17 | [symbol] | [symbol] | 1 | 1 |
| | | g1-related zinc finger protein | | | | | | | | | | | | | | | |
| [symbol] | [symbol] | arylsulfatase B isoform 1 precursor; N | [symbol] | [symbol] | 59687.2 | [symbol] | [symbol] | 0.99 | 0.68 | [symbol] | [symbol] | | | [symbol] | [symbol] | | |
| | | acetylgalactosamine-4-sulfatase | | | | | | | | | | | | | | | |
| [symbol] | [symbol] | galactin 3 binding protein; L3 antigen | [symbol] | [symbol] | 65331 | [symbol] | [symbol] | | 2.19 | [symbol] | [symbol] | | 27.8 | [symbol] | [symbol] | | 2 |
| | | Mac-2-binding protein; serum protein | | | | | | | | | | | | | | | |
| [symbol] | [symbol] | fibrinogen, beta chain preproprotein | [symbol] | [symbol] | 55902.1 | [symbol] | [symbol] | 0.9 | N/A | [symbol] | [symbol] | 23.7 | 27.2 | [symbol] | [symbol] | 1 | 1 |
| [symbol] | [symbol] | CD14 antigen precursor | [symbol] | [symbol] | 40076.2 | [symbol] | [symbol] | 0.96 | 1.09 | [symbol] | [symbol] | 20.3 | 18 | [symbol] | [symbol] | 4 | 2 |
| [symbol] | [symbol] | paraxonase 1; Paraxonase | [symbol] | [symbol] | 39731.3 | [symbol] | [symbol] | 0.94 | 0.95 | [symbol] | [symbol] | 29.9 | 26.1 | [symbol] | [symbol] | 3 | 3 |
| [symbol] | [symbol] | metalithionein 1A | [symbol] | [symbol] | 5133.3 | [symbol] | [symbol] | | N/A | [symbol] | [symbol] | | | [symbol] | [symbol] | | |
| [symbol] | [symbol] | cholesteryl ester transfer protein, plasma | [symbol] | [symbol] | 54770.2 | [symbol] | [symbol] | 0.99 | 1.14 | [symbol] | [symbol] | 17.5 | 23.4 | [symbol] | [symbol] | 2 | 2 |
| | | precursor | | | | | | | | | | | | | | | |
| [symbol] | [symbol] | hypothetical protein FLJ34064 | [symbol] | [symbol] | 97726.3 | [symbol] | [symbol] | 0.73 | N/A | [symbol] | [symbol] | | 23.1 | [symbol] | [symbol] | | 0 |
| [symbol] | [symbol] | Inter-alpha (globulin) inhibitor H4 (plasma | [symbol] | [symbol] | 103357.4 | [symbol] | [symbol] | | 1.12 | [symbol] | [symbol] | | | [symbol] | [symbol] | | |
| | | Kallikrein-sensitive glycoprotein); Inter- | | | | | | | | | | | | | | | |
| | | alpha (globulin) inhibitor. H polypeptide- | | | | | | | | | | | | | | | |
| | | like 1; inter-alpha (globulin) inhibitor, H4 | | | | | | | | | | | | | | | |
| | | polypeptide | | | | | | | | | | | | | | | |
| [symbol] | [symbol] | mitogen-activated protein kinase kinase | [symbol] | [symbol] | 81296.4 | [symbol] | [symbol] | 2 21 | 10.22 | [symbol] | [symbol] | 12.8 | | [symbol] | [symbol] | 2 | |
| | | kinase kinase 1; hematopoietic | | | | | | | | | | | | | | | |
| | | proganitor kinase 1 | | | | | | | | | | | | | | | |
| [symbol] | [symbol] | calcium binding protein 39-like | [symbol] | [symbol] | 33694.2 | [symbol] | [symbol] | | 1 | [symbol] | [symbol] | | 22.9 | [symbol] | [symbol] | | 3 |
| [symbol] | [symbol] | a disintegrin-like and metalloprotease | [symbol] | [symbol] | 214506.1 | [symbol] | [symbol] | 0.65 | 1.17 | [symbol] | [symbol] | 45.1 | 22.8 | [symbol] | [symbol] | 1 | 0 |
| | | (maprolysin type) with thrombospondin | | | | | | | | | | | | | | | |
| | | type 1 motif, 20 isoform 1; a disintegrin- | | | | | | | | | | | | | | | |
| | | like and metalloprotease with | | | | | | | | | | | | | | | |
| | | thrombospondin type 1 motifs 20 | | | | | | | | | | | | | | | |
| [symbol] | [symbol] | ephrin receptor EphB4 precursor; | [symbol] | [symbol] | 108270.2 | [symbol] | [symbol] | 0.98 | 0.29 | [symbol] | [symbol] | 13.7 | 22 | [symbol] | [symbol] | 0 | 0 |
| | | hapatonia transmembrane kinase | | | | | | | | | | | | | | | |
| [symbol] | [symbol] | PREDICTED: chromosome 8 open | [symbol] | [symbol] | 37270.1 | [symbol] | [symbol] | | 0.95 | [symbol] | [symbol] | | 21.8 | [symbol] | [symbol] | | 1 |
| | | reading frame 4 | | | | | | | | | | | | | | | |
| [symbol] | [symbol] | solute carrier family 37 member 1; | [symbol] | [symbol] | 57848.1 | [symbol] | [symbol] | | 0.99 | [symbol] | [symbol] | | 21.7 | [symbol] | [symbol] | | 1 |
| | | glycerol-3-phosphate permease | | | | | | | | | | | | | | | |
| [symbol] | [symbol] | zinc finger protein 568 | [symbol] | [symbol] | 43735.1 | [symbol] | [symbol] | 1.03 | 1.35 | [symbol] | [symbol] | 12.4 | | [symbol] | [symbol] | 1 | |
| [symbol] | [symbol] | gofgi autoantigen, golgin subfamily a, 4; | [symbol] | [symbol] | 261140.2 | [symbol] | [symbol] | 0.88 | 0.9 | [symbol] | [symbol] | 12.3 | 20.4 | [symbol] | [symbol] | 0 | 0 |
| | | gofgin-245; trans-Golgi p230; 256 kDa | | | | | | | | | | | | | | | |
| | | golgin: 72.1 protein; golgin-240 | | | | | | | | | | | | | | | |
| [symbol] | [symbol] | hypothetical protein BC008322 | [symbol] | [symbol] | 34854.5 | [symbol] | [symbol] | 2.11 | 0.9 | [symbol] | [symbol] | | 20 | [symbol] | [symbol] | | 4 |
| [symbol] | [symbol] | L-plastin; Lymphocyte cytomotic protein-1 | [symbol] | [symbol] | 70289.3 | [symbol] | [symbol] | 0.75 | 0.95 | [symbol] | [symbol] | | 19.3 | [symbol] | [symbol] | | 2 |
| | | (plasmin); plastin 2 | | | | | | | | | | | | | | | |
| [symbol] | [symbol] | serine (or cysteine) proteinase inhibitor, | [symbol] | [symbol] | 48738.6 | [symbol] | [symbol] | 1 | 0.88 | [symbol] | [symbol] | 20.3 | | [symbol] | [symbol] | 2 | |
| | | clade A (alpha-1 antiproteinase, | | | | | | | | | | | | | | | |
| | | antitrypsin), member 1; protease inhibitor | | | | | | | | | | | | | | | |
| | | 1 (anti-elastase), alpha-1-antitrypsin | | | | | | | | | | | | | | | |
| [symbol] | [symbol] | leucine-rich repeat-containing G protein- | [symbol] | [symbol] | 99266.8 | [symbol] | [symbol] | 1.01 | 0.95 | [symbol] | [symbol] | 27.8 | | [symbol] | [symbol] | 0 | |
| | | coupled receptor 6 | | | | | | | | | | | | | | | |
| [symbol] | [symbol] | plasma coagulation factor XI precursor | [symbol] | [symbol] | 70109.1 | [symbol] | [symbol] | 1 | 1.09 | [symbol] | [symbol] | 53.2 | | [symbol] | [symbol] | 8 | |
| | | isoform a; plasma thromboplastin | | | | | | | | | | | | | | | |
| | | antecadent | | | | | | | | | | | | | | | |
| [symbol] | [symbol] | serine (or cysteine) proteinase inhibitor, | [symbol] | [symbol] | 46542 | [symbol] | [symbol] | 1.04 | 0.89 | [symbol] | [symbol] | 12.8 | | [symbol] | [symbol] | 1 | |
| | | clade A (alpha-1 antiproteinase, | | | | | | | | | | | | | | | |
| | | antitrypsin), member 4; protease inhibitor | | | | | | | | | | | | | | | |
| | | 4 (kellistatin) | | | | | | | | | | | | | | | |
| [symbol] | [symbol] | transthyretin; prealbumin | [symbol] | [symbol] | 15887 | [symbol] | [symbol] | 1 | 1.35 | [symbol] | [symbol] | 27.8 | 14.3 | [symbol] | [symbol] | 4 | 4 |
| [symbol] | [symbol] | complement component 1, q | [symbol] | [symbol] | 25729.6 | [symbol] | [symbol] | 0.95 | 1.04 | [symbol] | [symbol] | 49.6 | 15.3 | [symbol] | [symbol] | 4 | 4 |
| | | subcomponent, gamma polypeptide; | | | | | | | | | | | | | | | |
| | | complement component C1q, C chain | | | | | | | | | | | | | | | |
| [symbol] | [symbol] | ubiquitin protein ligase E3 component n- | [symbol] | [symbol] | 200210.8 | [symbol] | [symbol] | 1.17 | 1.67 | [symbol] | [symbol] | | 12.5 | [symbol] | [symbol] | | 0 |
| | | recognin 1: ubiquitin ligase E3 alpha-I | | | | | | | | | | | | | | | |
| [symbol] | [symbol] | excision repair cross-complementing | [symbol] | [symbol] | 89277.7 | [symbol] | [symbol] | 0.75 | 1.09 | [symbol] | [symbol] | 24.8 | 14.9 | [symbol] | [symbol] | 1 | 1 |
| | | rodent repair deficiency, | | | | | | | | | | | | | | | |
| | | complementation group 3; xeroderma | | | | | | | | | | | | | | | |
| | | pigmentosum, complementation group 5 | | | | | | | | | | | | | | | |
| [symbol] | [symbol] | PREDICTED; similar to KIAA0446 | [symbol] | [symbol] | 296001.2 | [symbol] | [symbol] | 0.84 | 0.74 | [symbol] | [symbol] | 19.9 | | [symbol] | [symbol] | 0 | |
| [symbol] | [symbol] | T1 protein | [symbol] | [symbol] | 139422.9 | [symbol] | [symbol] | 0.94 | 1.41 | [symbol] | [symbol] | 13.3 | 13.7 | [symbol] | [symbol] | 0 | 0 |
| [symbol] | [symbol] | retinoblastoma-associated factor 800 | [symbol] | [symbol] | 573901 | [symbol] | [symbol] | 0.6 | N/A | [symbol] | [symbol] | 58.8 | | [symbol] | [symbol] | 0 | |
| [symbol] | [symbol] | PREDICTED: KIAA1083 protein | [symbol] | [symbol] | 188211.8 | [symbol] | [symbol] | N/A | 0.85 | [symbol] | [symbol] | 20.5 | 13 | [symbol] | [symbol] | 1 | 0 |
| [symbol] | [symbol] | PREDICTED: hypothetical protein | [symbol] | [symbol] | 29364.1 | [symbol] | [symbol] | N/A | 0.99 | [symbol] | [symbol] | 27.8 | 11.5 | [symbol] | [symbol] | 3 | 2 |
| [symbol] | [symbol] | zinc finger protein ZNF-U89274: zinc | [symbol] | [symbol] | 119382.6 | [symbol] | [symbol] | N/A | N/A | [symbol] | [symbol] | 15.8 | 11.2 | [symbol] | [symbol] | 0 | 0 |
| | | finger protein | | | | | | | | | | | | | | | |
| [symbol] | [symbol] | hypothetical protein FLJ36728 | [symbol] | [symbol] | 72363.7 | [symbol] | [symbol] | 0.82 | 1.19 | [symbol] | [symbol] | 25.1 | 10.7 | [symbol] | [symbol] | 2 | 1 |
| [symbol] | [symbol] | pregnancy zone protein; Pregnancy zone | [symbol] | [symbol] | 163835.9 | [symbol] | [symbol] | 1.02 | | [symbol] | [symbol] | | | [symbol] | [symbol] | | |
| | | protein | | | | | | | | | | | | | | | |
| [symbol] | [symbol] | altractin isoform 3; altractin-2; mahogany | [symbol] | [symbol] | 133701.6 | [symbol] | [symbol] | 0.9 | | [symbol] | [symbol] | | | [symbol] | [symbol] | | |
| | | protein | | | | | | | | | | | | | | | |
| [symbol] | [symbol] | mannan-binding lectin serine protease 1 | [symbol] | [symbol] | 81860.3 | [symbol] | [symbol] | 0.97 | | [symbol] | [symbol] | 135 | | [symbol] | [symbol] | 6 | |
| | | isoform 2, precursor, protease, serine, 5 | | | | | | | | | | | | | | | |
| | | (mannose-binding protein-associated); | | | | | | | | | | | | | | | |
| | | manan-binding lectin serine protease-1; | | | | | | | | | | | | | | | |
| | | Re-reactive factor serine protease p100 | | | | | | | | | | | | | | | |
| [symbol] | [symbol] | cartilage oligomeric matrix protein | [symbol] | [symbol] | 82880.5 | [symbol] | [symbol] | 1.22 | | [symbol] | [symbol] | 102.7 | | [symbol] | [symbol] | 7 | |
| | | precursor; epiphyseal dysplasia, multiple | | | | | | | | | | | | | | | |
| | | 1; pseudoachondroplasia (epiphyseal | | | | | | | | | | | | | | | |
| | | dysplasia 1, multiple); cartilage | | | | | | | | | | | | | | | |
| | | oligomeric matrix | | | | | | | | | | | | | | | |
| | | protein(pseudoachondroplasia, | | | | | | | | | | | | | | | |
| | | epiphyseal dysplasia 1, multiple); | | | | | | | | | | | | | | | |
| [symbol] | [symbol] | fibulln 1 isoform C precursor | [symbol] | [symbol] | 74461.9 | [symbol] | [symbol] | 0.78 | | [symbol] | [symbol] | 48.3 | | [symbol] | [symbol] | 2 | |
| [symbol] | [symbol] | lipoprotein. Lp(a); Apolipoprotein Lp(a); | [symbol] | [symbol] | 501319.1 | [symbol] | [symbol] | 1 | | [symbol] | [symbol] | 92.2 | | [symbol] | [symbol] | 1 | |
| | | antiangiogenic AK38 protein | | | | | | | | | | | | | | | |
| [symbol] | [symbol] | von Willebrand factor precursor: | [symbol] | [symbol] | 309298.6 | [symbol] | [symbol] | 1.09 | | [symbol] | [symbol] | 84.3 | | [symbol] | [symbol] | 1 | |
| | | Coagulation factor VIII VWF (von | | | | | | | | | | | | | | | |
| | | Willebrand factor) | | | | | | | | | | | | | | | |
| [symbol] | [symbol] | low density lipoprotein-related protein 1; | [symbol] | [symbol] | 504575.3 | [symbol] | [symbol] | 0.98 | | [symbol] | [symbol] | 42.9 | | [symbol] | [symbol] | 0 | |
| | | alpha-2-macroglobulin receptor | | | | | | | | | | | | | | | |
| [symbol] | [symbol] | procollagen C-endopeptidase enhancer; | [symbol] | [symbol] | 47946.4 | [symbol] | [symbol] | 1.38 | | [symbol] | [symbol] | 84.2 | | [symbol] | [symbol] | 6 | |
| | | procollagen, type 1, COOH-terminal | | | | | | | | | | | | | | | |
| | | proteinase enhancer | | | | | | | | | | | | | | | |
| [symbol] | [symbol] | plasminogen-related protein B; type B | [symbol] | [symbol] | 10970.5 | [symbol] | [symbol] | 0.99 | | [symbol] | [symbol] | | | [symbol] | [symbol] | | |
| | | plasminogen-related gene | | | | | | | | | | | | | | | |
| [symbol] | [symbol] | PREDICTED: kelch repeat and BTB | [symbol] | [symbol] | 631761.2 | [symbol] | [symbol] | 1.15 | | [symbol] | [symbol] | 79.4 | | [symbol] | [symbol] | 0 | |
| | | (POZ) domain containing 9 | | | | | | | | | | | | | | | |
| [symbol] | [symbol] | stabilin 2; CD44-like precursor FELL; | [symbol] | [symbol] | 276994.1 | [symbol] | [symbol] | N/A | | [symbol] | [symbol] | 14 | | [symbol] | [symbol] | 0 | |
| | | hyaluronan receptor for endocytosis | | | | | | | | | | | | | | | |
| [symbol] | [symbol] | low density lipoprotein-related protein 1B; | [symbol] | [symbol] | 515398.8 | [symbol] | [symbol] | 0.62 | | [symbol] | [symbol] | 66.4 | | [symbol] | [symbol] | 0 | |
| | | low density lipoprotein receptor related | | | | | | | | | | | | | | | |
| | | protein-deleted in tumor | | | | | | | | | | | | | | | |
| [symbol] | [symbol] | KIAA1404 protein | [symbol] | [symbol] | 220228.8 | [symbol] | [symbol] | 1.73 | | [symbol] | [symbol] | 46.1 | | [symbol] | [symbol] | 1 | |
| [symbol] | [symbol] | selectin L; lymph node horning receptor, | [symbol] | [symbol] | 42187.1 | [symbol] | [symbol] | 0.87 | | [symbol] | [symbol] | 58.3 | | [symbol] | [symbol] | 0 | |
| | | lymphocyte adhesion molecule 1 | | | | | | | | | | | | | | | |
| [symbol] | [symbol] | dynein, axonernal, heavy polpeptide 8 | [symbol] | [symbol] | 514819.9 | [symbol] | [symbol] | 1.04 | | [symbol] | [symbol] | 58.3 | | [symbol] | [symbol] | 1 | |
| [symbol] | [symbol] | FC fragment of IgG binding protein; IgG | [symbol] | [symbol] | 572096.7 | [symbol] | [symbol] | 1.24 | | [symbol] | [symbol] | 11 | | [symbol] | [symbol] | 0 | |
| | | Fc binding protein | | | | | | | | | | | | | | | |
| [symbol] | [symbol] | v-ski sarcoma viral oncogene homolg; | [symbol] | [symbol] | 80005.1 | [symbol] | [symbol] | 1.35 | | [symbol] | [symbol] | 52.8 | | [symbol] | [symbol] | 4 | |
| | | Avian sarcoma viral (v-ski) oncogene | | | | | | | | | | | | | | | |
| | | homolog; v-ski avian sarcoma viral | | | | | | | | | | | | | | | |
| | | oncogene homolog | | | | | | | | | | | | | | | |
| [symbol] | [symbol] | PREDICTED: similar to hypothetical | [symbol] | [symbol] | 39317 | [symbol] | [symbol] | 0.82 | | [symbol] | [symbol] | 49.1 | | [symbol] | [symbol] | 5 | |
| [symbol] | [symbol] | GREB1 protein Isoform a; gene regulated | [symbol] | [symbol] | 216467.3 | [symbol] | [symbol] | 1 | | [symbol] | [symbol] | 46 | | [symbol] | [symbol] | 1 | |
| | | by cetrogen in breast cancer protein | | | | | | | | | | | | | | | |
| [symbol] | [symbol] | hypothetical protein FLJ13908 | [symbol] | [symbol] | 28645.1 | [symbol] | [symbol] | N/A | | [symbol] | [symbol] | | | [symbol] | [symbol] | | |
| [symbol] | [symbol] | a disintegrin and metlioproteinase with | [symbol] | [symbol] | 216491.2 | [symbol] | [symbol] | 0.26 | | [symbol] | [symbol] | 45.7 | | [symbol] | [symbol] | 1 | |
| | | thrombospondin motifs 9 isoform 1 | | | | | | | | | | | | | | | |
| | | preproprotein | | | | | | | | | | | | | | | |
| [symbol] | [symbol] | DNA (cytosine-5-)-methyltransferase 1; | [symbol] | [symbol] | 183165.2 | [symbol] | [symbol] | 0.6 | | [symbol] | [symbol] | 45.5 | | [symbol] | [symbol] | 0 | |
| | | DNA methyltransferase; DNA | | | | | | | | | | | | | | | |
| | | methyltransferase 1 | | | | | | | | | | | | | | | |
| [symbol] | [symbol] | hypothetical protein LOC129607 | [symbol] | [symbol] | 32645.7 | [symbol] | [symbol] | 0.72 | | [symbol] | [symbol] | 44.8 | | [symbol] | [symbol] | 6 | |
| [symbol] | [symbol] | ubiquitous tetratricopeptide containing | [symbol] | [symbol] | 109658 | [symbol] | [symbol] | 0.66 | | [symbol] | [symbol] | 44.2 | | [symbol] | [symbol] | 2 | |
| | | protein RoXaN; Rotavirus ‘X’ associated | | | | | | | | | | | | | | | |
| | | non-structural protein | | | | | | | | | | | | | | | |
| [symbol] | [symbol] | scavenger receptor Cysteine-rich type 1 | [symbol] | [symbol] | 158257.1 | [symbol] | [symbol] | N/A | | [symbol] | [symbol] | 44.1 | | [symbol] | [symbol] | 3 | |
| | | protein M160 precursor; CD163 antigen | | | | | | | | | | | | | | | |
| [symbol] | [symbol] | integrin beta chain, beta 2 precursor, | [symbol] | [symbol] | 84790.8 | [symbol] | [symbol] | 1.48 | | [symbol] | [symbol] | 43.9 | | [symbol] | [symbol] | 3 | |
| | | integrin, beta-2 (antigen CD18 (p95), | | | | | | | | | | | | | | | |
| | | lymphocyte function-associated; cell | | | | | | | | | | | | | | | |
| | | surface adhesion glycoprotein (LFA- | | | | | | | | | | | | | | | |
| | | 1/CR3/P150, 959 beta subunit precursor) | | | | | | | | | | | | | | | |
| [symbol] | [symbol] | fibrinogen, gamma chain isoform | [symbol] | [symbol] | 49481.5 | [symbol] | [symbol] | 1.1 | | [symbol] | [symbol] | 19.2 | | [symbol] | [symbol] | 4 | |
| | | gamma-A precursor | | | | | | | | | | | | | | | |
| [symbol] | [symbol] | PREDICTED: hypothetical protein | [symbol] | [symbol] | 18929.2 | [symbol] | [symbol] | N/A | | [symbol] | [symbol] | 42 | | [symbol] | [symbol] | 7 | |
| [symbol] | [symbol] | PREDICTED: similar to RIKEN cDNA | [symbol] | [symbol] | 142106.7 | [symbol] | [symbol] | 1.03 | | [symbol] | [symbol] | 41.5 | | [symbol] | [symbol] | 0 | |
| | | 5430400H23 | | | | | | | | | | | | | | | |
| [symbol] | [symbol] | protein kinase, lysine deficient 1; kinase | [symbol] | [symbol] | 260765.7 | [symbol] | [symbol] | 1.18 | | [symbol] | [symbol] | 40.1 | | [symbol] | [symbol] | 0 | |
| | | delicient protein | | | | | | | | | | | | | | | |
| [symbol] | [symbol] | transmembrane protease, serine 6; | [symbol] | [symbol] | 90000.1 | [symbol] | [symbol] | 0.89 | | [symbol] | [symbol] | 40 | | [symbol] | [symbol] | 2 | |
| | | membrane-bound mosaic sarine | | | | | | | | | | | | | | | |
| | | proteinase mairplase-2; transmembrane | | | | | | | | | | | | | | | |
| | | serine protease 8; type II transmembrane | | | | | | | | | | | | | | | |
| | | serine protease 6 | | | | | | | | | | | | | | | |
| [symbol] | [symbol] | zinc finger protein 560 | [symbol] | [symbol] | 91135 | [symbol] | [symbol] | 1.01 | | [symbol] | [symbol] | 39.3 | | [symbol] | [symbol] | 1 | |
| [symbol] | [symbol] | factor H-related protein 5 | [symbol] | [symbol] | 64419.4 | [symbol] | [symbol] | 0.43 | | [symbol] | [symbol] | 38.8 | | [symbol] | [symbol] | 1 | |
| [symbol] | [symbol] | prenylcysteine oxidase 1; prenylcysteine | [symbol] | [symbol] | 58700.2 | [symbol] | [symbol] | 0.98 | | [symbol] | [symbol] | 36.8 | | [symbol] | [symbol] | 1 | |
| | | lyase | | | | | | | | | | | | | | | |
| [symbol] | [symbol] | zinc finger protein 625 | [symbol] | [symbol] | 34746.3 | [symbol] | [symbol] | 2.17 | | [symbol] | [symbol] | 17.1 | | [symbol] | [symbol] | 4 | |
| [symbol] | [symbol] | hypothetical protein FLJ32954 | [symbol] | [symbol] | 65228 | [symbol] | [symbol] | 0.4 | | [symbol] | [symbol] | 38.2 | | [symbol] | [symbol] | 2 | |
| [symbol] | [symbol] | similar to Hypothetical zinc finger protein | [symbol] | [symbol] | 63463.3 | [symbol] | [symbol] | N/A | | [symbol] | [symbol] | | | [symbol] | [symbol] | | |
| | | KIAA1559 | | | | | | | | | | | | | | | |
| [symbol] | [symbol] | guarrytale binding protein 4-like | [symbol] | [symbol] | 72525.3 | [symbol] | [symbol] | N/A | | [symbol] | [symbol] | 37.9 | | [symbol] | [symbol] | 3 | |
| [symbol] | [symbol] | zinc finger protein 521; early | [symbol] | [symbol] | 147886.1 | [symbol] | [symbol] | N/A | | [symbol] | [symbol] | | | [symbol] | [symbol] | | |
| | | hematopalatic zinc finger | | | | | | | | | | | | | | | |
| [symbol] | [symbol] | PREDICTED: chromosome 20 open | [symbol] | [symbol] | 90734.9 | [symbol] | [symbol] | 1.36 | | [symbol] | [symbol] | | | [symbol] | [symbol] | | |
| | | reading frame 82 | | | | | | | | | | | | | | | |
| [symbol] | [symbol] | smooth muscle myosin heavy chain 11 | [symbol] | [symbol] | 223577.3 | [symbol] | [symbol] | 0.8 | | [symbol] | [symbol] | 35.1 | | [symbol] | [symbol] | 1 | |
| | | isoform SM2 | | | | | | | | | | | | | | | |
| [symbol] | [symbol] | hypothetical protein SB153 isoform 2 | [symbol] | [symbol] | 33050.1 | [symbol] | [symbol] | 1.43 | | [symbol] | [symbol] | | | [symbol] | [symbol] | | |
| [symbol] | [symbol] | phospholinositide-3-kinase, class 2, alpha | [symbol] | [symbol] | 190737.7 | [symbol] | [symbol] | N/A | | [symbol] | [symbol] | 35.6 | | [symbol] | [symbol] | 1 | |
| | | polypeptide; C2-containing | | | | | | | | | | | | | | | |
| | | phosphalidylinositol kinase; PI3K- | | | | | | | | | | | | | | | |
| [symbol] | [symbol] | immunoglobulin superfamily, member 10 | [symbol] | [symbol] | 290837.9 | [symbol] | [symbol] | 0.81 | | [symbol] | [symbol] | | | [symbol] | [symbol] | | |
| [symbol] | [symbol] | myotonic dyatrophy protein kinase like | [symbol] | [symbol] | 172518.3 | [symbol] | [symbol] | 0.94 | | [symbol] | [symbol] | 21.8 | | [symbol] | [symbol] | 2 | |
| | | protein; protein kinase | | | | | | | | | | | | | | | |
| [symbol] | [symbol] | complement factor D preproprotein; | [symbol] | [symbol] | 27032.9 | [symbol] | [symbol] | 0.82 | | [symbol] | [symbol] | 22.7 | | [symbol] | [symbol] | 8 | |
| | | adipsin; propendin factor D; C3 | | | | | | | | | | | | | | | |
| [symbol] | [symbol] | zinc finger protein 592 | [symbol] | [symbol] | 137555.2 | [symbol] | [symbol] | 1.13 | | [symbol] | [symbol] | 34.4 | | [symbol] | [symbol] | 2 | |
| [symbol] | [symbol] | “NOV1” | [symbol] | [symbol] | 24619.7 | [symbol] | [symbol] | 1.53 | | [symbol] | [symbol] | 34.1 | | [symbol] | [symbol] | 5 | |
| [symbol] | [symbol] | multiple inositol polyphosphate histidine | [symbol] | [symbol] | 55051.2 | [symbol] | [symbol] | 0.93 | | [symbol] | [symbol] | 34.1 | | [symbol] | [symbol] | 1 | |
| | | phosphatase 1; multiple inositol | | | | | | | | | | | | | | | |
| | | polyphosphate phosphatase 2; multiple | | | | | | | | | | | | | | | |
| | | inositol polyphosphate phosphatase 1 | | | | | | | | | | | | | | | |
| [symbol] | [symbol] | KIAA1985 protein | [symbol] | [symbol] | 144776.6 | [symbol] | [symbol] | 2.04 | | [symbol] | [symbol] | 34.1 | | [symbol] | [symbol] | 1 | |
| [symbol] | [symbol] | insulin-like growth factor 2 (somatomedin | [symbol] | [symbol] | 20140.3 | [symbol] | [symbol] | 0.8 | | [symbol] | [symbol] | 38.7 | | [symbol] | [symbol] | 5 | |
| | | A); somatomedin A | | | | | | | | | | | | | | | |
| [symbol] | [symbol] | WINS1 protein isoform 2 | [symbol] | [symbol] | 49325 | [symbol] | [symbol] | 0.93 | | [symbol] | [symbol] | 33.7 | | [symbol] | [symbol] | 1 | |
| [symbol] | [symbol] | v-lof Hardy-Zuclerman 4 feline sarcoma | [symbol] | [symbol] | 109664.6 | [symbol] | [symbol] | 2 | | [symbol] | [symbol] | 33.4 | | [symbol] | [symbol] | 2 | |
| | | viral oncogene homolog precursor | | | | | | | | | | | | | | | |
| [symbol] | [symbol] | hyrosine kinase 2 | [symbol] | [symbol] | 133665.9 | [symbol] | [symbol] | N/A | | [symbol] | [symbol] | | | [symbol] | [symbol] | | |
| [symbol] | [symbol] | KIAA1729 protein | [symbol] | [symbol] | 119531.2 | [symbol] | [symbol] | N/A | | [symbol] | [symbol] | | | [symbol] | [symbol] | | |
| [symbol] | [symbol] | zinc finger protein 261 | [symbol] | [symbol] | 152379.1 | [symbol] | [symbol] | 1.25 | | [symbol] | [symbol] | | | [symbol] | [symbol] | | |
| [symbol] | [symbol] | zinc finger protein KIAA0961 | [symbol] | [symbol] | 61557.5 | [symbol] | [symbol] | N/A | | [symbol] | [symbol] | | | [symbol] | [symbol] | | |
| [symbol] | [symbol] | PREDICTED: zinc finger CCCH type | [symbol] | [symbol] | 108458.6 | [symbol] | [symbol] | 1.12 | | [symbol] | [symbol] | 32 | | [symbol] | [symbol] | 3 | |
| | | domain containing 5 | | | | | | | | | | | | | | | |
| [symbol] | [symbol] | mannose receptor, C type 2; andocytic | [symbol] | [symbol] | 186655.4 | [symbol] | [symbol] | 2.98 | | [symbol] | [symbol] | 12.8 | | [symbol] | [symbol] | 1 | |
| | | receptor (macrophage mannose receptor | | | | | | | | | | | | | | | |
| | | family); urokinase plasminogen activator | | | | | | | | | | | | | | | |
| | | receptor-associated protein | | | | | | | | | | | | | | | |
| [symbol] | [symbol] | zinc finger, SWTM domain containlng 4 | [symbol] | [symbol] | 110138 | [symbol] | [symbol] | N/A | | [symbol] | [symbol] | 31.7 | | [symbol] | [symbol] | 1 | |
| [symbol] | [symbol] | PREDICTED: hypothetical protein | [symbol] | [symbol] | 11479 | [symbol] | [symbol] | 0.88 | | [symbol] | [symbol] | 31.4 | | [symbol] | [symbol] | 8 | |
| [symbol] | [symbol] | PREDICTED: hypothetical protein | [symbol] | [symbol] | 22383.2 | [symbol] | [symbol] | N/A | | [symbol] | [symbol] | 31.3 | | [symbol] | [symbol] | 6 | |
| [symbol] | [symbol] | complement factor H-related 4 | [symbol] | [symbol] | 37325 | [symbol] | [symbol] | 0.88 | | [symbol] | [symbol] | 31.1 | | [symbol] | [symbol] | 3 | |
| [symbol] | [symbol] | spectrin, beta, non-erythrocylic 5: beta V | [symbol] | [symbol] | 416835.1 | [symbol] | [symbol] | N/A | | [symbol] | [symbol] | | | [symbol] | [symbol] | | |
| | | spectrin | | | | | | | | | | | | | | | |
| [symbol] | [symbol] | KIAAD676 protein isoform [symbol] | [symbol] | [symbol] | 140624.7 | [symbol] | [symbol] | 0.82 | | [symbol] | [symbol] | | | [symbol] | [symbol] | | |
| [symbol] | [symbol] | aquaporin 3 | [symbol] | [symbol] | 31543.8 | [symbol] | [symbol] | N/A | | [symbol] | [symbol] | | | [symbol] | [symbol] | | |
| [symbol] | [symbol] | somaphorin 4D; sama domain, | [symbol] | [symbol] | 96207.9 | [symbol] | [symbol] | 1.08 | | [symbol] | [symbol] | 29.6 | | [symbol] | [symbol] | 0 | |
| | | Immunoglobulin domain (lg). | | | | | | | | | | | | | | | |
| | | transmembrane domain (TM) and short | | | | | | | | | | | | | | | |
| | | cytoplasmic domain, 4D | | | | | | | | | | | | | | | |
| [symbol] | [symbol] | hypothetical protein MGC34032 | [symbol] | [symbol] | 81743.3 | [symbol] | [symbol] | 0.94 | | [symbol] | [symbol] | | | [symbol] | [symbol] | | |
| [symbol] | [symbol] | PREDICTED: similar to KIAA0033 | [symbol] | [symbol] | 70600.3 | [symbol] | [symbol] | N/A | | [symbol] | [symbol] | 20.6 | | [symbol] | [symbol] | 1 | |
| [symbol] | [symbol] | [symbol] csln | [symbol] | [symbol] | 504587.3 | [symbol] | [symbol] | 0.38 | | [symbol] | [symbol] | 28.8 | | [symbol] | [symbol] | 0 | |
| [symbol] | [symbol] | wingless-type MMTV integration site | [symbol] | [symbol] | 39000.8 | [symbol] | [symbol] | N/A | | [symbol] | [symbol] | 28.4 | | [symbol] | [symbol] | 2 | |
| | | family. member 9B precursor. wingless- | | | | | | | | | | | | | | | |
| | | type MMTV integration site family, | | | | | | | | | | | | | | | |
| [symbol] | [symbol] | tripartite motif protein 15 Isoform beta | [symbol] | [symbol] | 12301.1 | [symbol] | [symbol] | 0.88 | | [symbol] | [symbol] | 11.2 | | [symbol] | [symbol] | 8 | |
| [symbol] | [symbol] | proteasome alpha 7 subunit isoform 1; | [symbol] | [symbol] | 27586.8 | [symbol] | [symbol] | 1.76 | | [symbol] | [symbol] | | | [symbol] | [symbol] | | |
| | | proteasome subunit RC8-1; proteasome | | | | | | | | | | | | | | | |
| | | subunit XAPC7 | | | | | | | | | | | | | | | |
| [symbol] | [symbol] | winglass-type MMTV Integration site | [symbol] | [symbol] | 46444.3 | [symbol] | [symbol] | 0.66 | | [symbol] | [symbol] | 27 | | [symbol] | [symbol] | 5 | |
| | | family, member 10A precursor | | | | | | | | | | | | | | | |
| [symbol] | [symbol] | gp130-like monocyte receptor; soluble | [symbol] | [symbol] | 82953.8 | [symbol] | [symbol] | 0.74 | | [symbol] | [symbol] | 27 | | [symbol] | [symbol] | 1 | |
| | | type I cytokine receptor CRL3; GP130 | | | | | | | | | | | | | | | |
| | | like receptor | | | | | | | | | | | | | | | |
| [symbol] | [symbol] | 5100 calcium-binding protein A8; | [symbol] | [symbol] | 10834.5 | [symbol] | [symbol] | 0.71 | | [symbol] | [symbol] | | | [symbol] | [symbol] | | |
| | | calgranulin A; cysile librosis antigen; | | | | | | | | | | | | | | | |
| | | 8100 calcium-binding protein A8 | | | | | | | | | | | | | | | |
| [symbol] | [symbol] | extracellular matrix protein 2 | [symbol] | [symbol] | 79789.3 | [symbol] | [symbol] | N/A | | [symbol] | [symbol] | | | [symbol] | [symbol] | | |
| [symbol] | [symbol] | kinase insert domain receptor (a type III | [symbol] | [symbol] | 151526.8 | [symbol] | [symbol] | 2.03 | | [symbol] | [symbol] | | | [symbol] | [symbol] | | |
| | | receptor tyrosine kinase); Kinase insert | | | | | | | | | | | | | | | |
| | | domain receptor | | | | | | | | | | | | | | | |
| [symbol] | [symbol] | PR domain containing 11; PR-domain | [symbol] | [symbol] | 57862.9 | [symbol] | [symbol] | 0.77 | | [symbol] | [symbol] | | | [symbol] | [symbol] | | |
| | | containing protein 11 | | | | | | | | | | | | | | | |
| [symbol] | [symbol] | hypothetical protein FLJ14936 | [symbol] | [symbol] | 37476.5 | [symbol] | [symbol] | N/A | | [symbol] | [symbol] | | | [symbol] | [symbol] | | |
| [symbol] | [symbol] | zinc finger protein 177 | [symbol] | [symbol] | 36473.1 | [symbol] | [symbol] | 1.12 | | [symbol] | [symbol] | 26 | | [symbol] | [symbol] | 3 | |
| [symbol] | [symbol] | neuroplastoma-amplified protein | [symbol] | [symbol] | 268571.3 | [symbol] | [symbol] | 0.96 | | [symbol] | [symbol] | | | [symbol] | [symbol] | | |
| [symbol] | [symbol] | gonadolrapin inducible transcription | [symbol] | [symbol] | 88214 | [symbol] | [symbol] | 0.97 | | [symbol] | [symbol] | | | [symbol] | [symbol] | | |
| | | repressor 1 | | | | | | | | | | | | | | | |
| [symbol] | [symbol] | a disintegrin-like and metalloprotease | [symbol] | [symbol] | 136167.1 | [symbol] | [symbol] | N/A | | [symbol] | [symbol] | 25.4 | | [symbol] | [symbol] | 1 | |
| | | (reprolysin type) with thrombospondin | | | | | | | | | | | | | | | |
| | | type 1 motif, 18 isoform 1 preproprotein, | | | | | | | | | | | | | | | |
| | | a disintegrin-like and metalloprotease | | | | | | | | | | | | | | | |
| | | (reprolysin type) with thromboepondin | | | | | | | | | | | | | | | |
| | | type 1 motiff, 21 | | | | | | | | | | | | | | | |
| [symbol] | [symbol] | thromboepondin type \| domain-containing | [symbol] | [symbol] | 94683.9 | [symbol] | [symbol] | 0.86 | | [symbol] | [symbol] | 25.4 | | [symbol] | [symbol] | 1 | |
| | | 1 isoform 1; 4833423O18Rlk; | | | | | | | | | | | | | | | |
| | | transmembrane molecule with | | | | | | | | | | | | | | | |
| | | thromboepondin molecule; | | | | | | | | | | | | | | | |
| [symbol] | [symbol] | tarsh protein | [symbol] | [symbol] | 118642 | [symbol] | [symbol] | 0.88 | | [symbol] | [symbol] | 24.7 | | [symbol] | [symbol] | 1 | |
| [symbol] | [symbol] | rhoinbold, veinlet-like 4; ventrifold | [symbol] | [symbol] | 45244.6 | [symbol] | [symbol] | 0.33 | | [symbol] | [symbol] | 25.1 | | [symbol] | [symbol] | 4 | |
| | | transmembrane protein | | | | | | | | | | | | | | | |
| [symbol] | [symbol] | glutamate recaptor KA1 precursor, | [symbol] | [symbol] | 107245.5 | [symbol] | [symbol] | N/A | | [symbol] | [symbol] | | | [symbol] | [symbol] | | |
| | | excitalory amino acid receptor 1 | | | | | | | | | | | | | | | |
| [symbol] | [symbol] | splicing factor, arginine-serine-rich 5 | [symbol] | [symbol] | 12626 | [symbol] | [symbol] | 0.76 | | [symbol] | [symbol] | 24.8 | | [symbol] | [symbol] | 9 | |
| [symbol] | [symbol] | PREDICTED: similar to Afaxin-1 | [symbol] | [symbol] | 70403.9 | [symbol] | [symbol] | N/A | | [symbol] | [symbol] | 24.8 | | [symbol] | [symbol] | 2 | |
| | | (Spinocarebeller alaxis type 1 protein | | | | | | | | | | | | | | | |
| | | homolog) | | | | | | | | | | | | | | | |
| [symbol] | [symbol] | heat shock 90 kDa protein 1, alpha; heat | [symbol] | [symbol] | 64673.7 | [symbol] | [symbol] | 0.91 | | [symbol] | [symbol] | 24.7 | | [symbol] | [symbol] | 0 | |
| | | shock 90 kDa protein 1, alpha | | | | | | | | | | | | | | | |
| [symbol] | [symbol] | PREDICTED: similar to carbonic | [symbol] | [symbol] | 70702.3 | [symbol] | [symbol] | N/A | | [symbol] | [symbol] | 18 | | [symbol] | [symbol] | 2 | |
| | | anhydrase VA, mitochondrial precursor; | | | | | | | | | | | | | | | |
| | | carbonic anhydrase V, mitochondrial, | | | | | | | | | | | | | | | |
| | | carbonic dehydralase | | | | | | | | | | | | | | | |
| [symbol] | [symbol] | PREDICTED: myosin VB | [symbol] | [symbol] | 266807.1 | [symbol] | [symbol] | 3.2 | | [symbol] | [symbol] | 24.5 | | [symbol] | [symbol] | 0 | |
| [symbol] | [symbol] | hypothetical protein FLJ14788 | [symbol] | [symbol] | 51893.9 | [symbol] | [symbol] | N/A | | [symbol] | [symbol] | 24.2 | | [symbol] | [symbol] | 3 | |
| [symbol] | [symbol] | hypothetical protein DT1P1A10 | [symbol] | [symbol] | 20894 | [symbol] | [symbol] | N/A | | [symbol] | [symbol] | 24.2 | | [symbol] | [symbol] | 4 | |
| [symbol] | [symbol] | signal transducer and activator of | [symbol] | [symbol] | 90647 | [symbol] | [symbol] | N/A | | [symbol] | [symbol] | 24 | | [symbol] | [symbol] | 0 | |
| | | transcription 6A | | | | | | | | | | | | | | | |
| [symbol] | [symbol] | thyroid peroxidase isoform a; | [symbol] | [symbol] | 102982.7 | [symbol] | [symbol] | 1.34 | | [symbol] | [symbol] | 12.2 | | [symbol] | [symbol] | 1 | |
| | | thyroperoxidase: thyroid microsomal | | | | | | | | | | | | | | | |
| | | antigen | | | | | | | | | | | | | | | |
| [symbol] | [symbol] | DEAH (Asp-Glu-His) box polypeptide | [symbol] | [symbol] | 78874.1 | [symbol] | [symbol] | N/A | | [symbol] | [symbol] | | | [symbol] | [symbol] | | |
| | | 33; DEADH (Asp-Glu-Ala-Asp/His) box | | | | | | | | | | | | | | | |
| | | polypeptide 33 | | | | | | | | | | | | | | | |
| [symbol] | [symbol] | protein phosperilase 5, catalytic subunit | [symbol] | [symbol] | 58878.6 | [symbol] | [symbol] | N/A | | [symbol] | [symbol] | | | [symbol] | [symbol] | | |
| [symbol] | [symbol] | PREDICTED; KIAA1447 protein | [symbol] | [symbol] | 269382.4 | [symbol] | [symbol] | 0.81 | | [symbol] | [symbol] | | | [symbol] | [symbol] | | |
| [symbol] | [symbol] | HLA-B associated transcript 5; HLA-B | [symbol] | [symbol] | 83243.8 | [symbol] | [symbol] | 1.06 | | [symbol] | [symbol] | 23.8 | | [symbol] | [symbol] | 2 | |
| | | associated transcript-5; BAT5 protein | | | | | | | | | | | | | | | |
| [symbol] | [symbol] | interferon-alpha receptor 1 precursor; | [symbol] | [symbol] | 63525.3 | [symbol] | [symbol] | N/A | | [symbol] | [symbol] | | | [symbol] | [symbol] | | |
| | | alpha-type antiviral protein; beta-type | | | | | | | | | | | | | | | |
| | | antiviral protein; interferon-beta receptor | | | | | | | | | | | | | | | |
| | | 1; interferon-alpha/beta receptor alpha | | | | | | | | | | | | | | | |
| [symbol] | [symbol] | PREDICTED: similar to 33 kDa protein | [symbol] | [symbol] | 40917.2 | [symbol] | [symbol] | 0.78 | | [symbol] | [symbol] | 23.7 | | [symbol] | [symbol] | 1 | |
| [symbol] | [symbol] | neuralized-like protein 2; neuralized-like | [symbol] | [symbol] | 31689.6 | [symbol] | [symbol] | N/A | | [symbol] | [symbol] | | | [symbol] | [symbol] | | |
| | | 2; chromosome 20 open reading frame | | | | | | | | | | | | | | | |
| [symbol] | [symbol] | solute carrier family 29 (nucleoside | [symbol] | [symbol] | 58114.7 | [symbol] | [symbol] | 0.96 | | [symbol] | [symbol] | 23.6 | | [symbol] | [symbol] | 3 | |
| | | transporters), member 4; aquilibrative | | | | | | | | | | | | | | | |
| | | nucleoside transporter 4 | | | | | | | | | | | | | | | |
| [symbol] | [symbol] | PREDICTED: DNA2 DNA replication | [symbol] | [symbol] | 129681.5 | [symbol] | [symbol] | 0.57 | | [symbol] | [symbol] | 12.4 | | [symbol] | [symbol] | 1 | |
| | | helicase 2-like | | | | | | | | | | | | | | | |
| [symbol] | [symbol] | vanin 1 precursor, Vannin 1; | [symbol] | [symbol] | 67023.7 | [symbol] | [symbol] | 0.67 | | [symbol] | [symbol] | 23.5 | | [symbol] | [symbol] | 2 | |
| [symbol] | [symbol] | neuraxin 1 isoform alpha precursor; | [symbol] | [symbol] | 161882.9 | [symbol] | [symbol] | N/A | | [symbol] | [symbol] | 23.5 | | [symbol] | [symbol] | 1 | |
| | | neuraxin 1 | | | | | | | | | | | | | | | |
| [symbol] | [symbol] | coronin, actin binding protein, 1A; | [symbol] | [symbol] | 51026.3 | [symbol] | [symbol] | N/A | | [symbol] | [symbol] | 23.5 | | [symbol] | [symbol] | 2 | |
| | | coronin, actin-binding, 1A; coronin, actin- | | | | | | | | | | | | | | | |
| | | binding protein, 1A; coronin-1 | | | | | | | | | | | | | | | |
| [symbol] | [symbol] | similar to 60S ribosomal protein L10(QM | [symbol] | [symbol] | 24626.9 | [symbol] | [symbol] | N/A | | [symbol] | [symbol] | 23.3 | | [symbol] | [symbol] | 4 | |
| | | protein homolog) | | | | | | | | | | | | | | | |
| [symbol] | [symbol] | chromosome 20 open reading frame 42; | [symbol] | [symbol] | 77408.8 | [symbol] | [symbol] | N/A | | [symbol] | [symbol] | 23.2 | | [symbol] | [symbol] | 0 | |
| | | UNC-112 related protein 1; kindlin 1; | | | | | | | | | | | | | | | |
| | | kindlerin | | | | | | | | | | | | | | | |
| [symbol] | [symbol] | PREDICTED: bicaudal C homolog 1 | [symbol] | [symbol] | 53276.3 | [symbol] | [symbol] | N/A | | [symbol] | [symbol] | 23.2 | | [symbol] | [symbol] | 3 | |
| [symbol] | [symbol] | PREDICTED: similar to Charot-Layden | [symbol] | [symbol] | 16189.4 | [symbol] | [symbol] | N/A | | [symbol] | [symbol] | 17.3 | | [symbol] | [symbol] | 6 | |
| | | crystal protein; osinophil | | | | | | | | | | | | | | | |
| | | lysophospholpase; lysolacthin | | | | | | | | | | | | | | | |
| | | acylhydradase; gatactin-10 | | | | | | | | | | | | | | | |
| [symbol] | [symbol] | hypothetical protein FLJ10863 | [symbol] | [symbol] | 16473.8 | [symbol] | [symbol] | N/A | | [symbol] | [symbol] | | | [symbol] | [symbol] | | |
| [symbol] | [symbol] | hypothetical protein XP_376795 | [symbol] | [symbol] | 19258.2 | [symbol] | [symbol] | 0.88 | | [symbol] | [symbol] | 22.9 | | [symbol] | [symbol] | 7 | |
| [symbol] | [symbol] | PREDICTED: similar to ankylin repeat | [symbol] | [symbol] | 60218.7 | [symbol] | [symbol] | 0.82 | | [symbol] | [symbol] | | | [symbol] | [symbol] | | |
| | | domain 20A | | | | | | | | | | | | | | | |
| [symbol] | [symbol] | Interleukin 19 isoform 2 precursor; | [symbol] | [symbol] | 20451.8 | [symbol] | [symbol] | N/A | | [symbol] | [symbol] | 22.6 | | [symbol] | [symbol] | 5 | |
| | | melanoma differentiation associated | | | | | | | | | | | | | | | |
| | | protein-like protein | | | | | | | | | | | | | | | |
| [symbol] | [symbol] | axostosin 1 | [symbol] | [symbol] | 86254.8 | [symbol] | [symbol] | N/A | | [symbol] | [symbol] | | | [symbol] | [symbol] | | |
| [symbol] | [symbol] | solute carrier organic anion transporter | [symbol] | [symbol] | 77193.3 | [symbol] | [symbol] | 0.65 | | [symbol] | [symbol] | 22.7 | | [symbol] | [symbol] | 0 | |
| | | family member 4A1; solute carrier family | | | | | | | | | | | | | | | |
| | | 21 member 12; organic anion | | | | | | | | | | | | | | | |
| | | transporting polypeptide E; sodium- | | | | | | | | | | | | | | | |
| | | independent organic anion transporter E; | | | | | | | | | | | | | | | |
| | | organic anion transporter polypeptide- | | | | | | | | | | | | | | | |
| | | related protein 1; colon organi | | | | | | | | | | | | | | | |
| [symbol] | [symbol] | PREDICTED: similar to P38IP protein | [symbol] | [symbol] | 93655 | [symbol] | [symbol] | N/A | | [symbol] | [symbol] | 22.6 | | [symbol] | [symbol] | 0 | |
| [symbol] | [symbol] | connector enhancer ol kinase | [symbol] | [symbol] | 117534.5 | [symbol] | [symbol] | N/A | | [symbol] | [symbol] | | | [symbol] | [symbol] | | |
| | | suppressor of Ras 2; connector enhancer | | | | | | | | | | | | | | | |
| [symbol] | [symbol] | Inonitol 1,4,5-triphosphate receptor, type | [symbol] | [symbol] | 306773.3 | [symbol] | [symbol] | 0.99 | | [symbol] | [symbol] | 21.5 | | [symbol] | [symbol] | 0 | |
| [symbol] | [symbol] | surfactant, pulmonary-associated protein | [symbol] | [symbol] | 26169.3 | [symbol] | [symbol] | 0.95 | | [symbol] | [symbol] | 22.6 | | [symbol] | [symbol] | 2 | |
| | | AZ | | | | | | | | | | | | | | | |
| [symbol] | [symbol] | PREDICTED similar to Dual specifity | [symbol] | [symbol] | 38366.5 | [symbol] | [symbol] | 0.98 | | [symbol] | [symbol] | 22.3 | | [symbol] | [symbol] | 2 | |
| | | protein phosphatase 13 (Tests-and | | | | | | | | | | | | | | | |
| | | skelatal-muscle-specific DSP) | | | | | | | | | | | | | | | |
| [symbol] | [symbol] | mitochondrial ribosomal protein L45 | [symbol] | [symbol] | 35242.8 | [symbol] | [symbol] | 0.98 | | [symbol] | [symbol] | 22.1 | | [symbol] | [symbol] | 4 | |
| [symbol] | [symbol] | trypothetical protein FLJ20438 | [symbol] | [symbol] | 43483 | [symbol] | [symbol] | N/A | | [symbol] | [symbol] | | | [symbol] | [symbol] | | |
| [symbol] | [symbol] | sarF domain containing kinase 6 | [symbol] | [symbol] | 55896.7 | [symbol] | [symbol] | 0.84 | | [symbol] | [symbol] | 21.9 | | [symbol] | [symbol] | 3 | |
| [symbol] | [symbol] | heparan sullfate 5-O-sulfotransferance; | [symbol] | [symbol] | 47075.8 | [symbol] | [symbol] | N/A | | [symbol] | [symbol] | 21.9 | | [symbol] | [symbol] | 2 | |
| | | heparan-sulfate 6-sulfotransferase | | | | | | | | | | | | | | | |
| [symbol] | [symbol] | rab-related GTP-binding protein | [symbol] | [symbol] | 25006.7 | [symbol] | [symbol] | 0.99 | | [symbol] | [symbol] | 21.8 | | [symbol] | [symbol] | 2 | |
| [symbol] | [symbol] | helecase/primase complex protein | [symbol] | [symbol] | 27664.6 | [symbol] | [symbol] | 0.81 | | [symbol] | [symbol] | | | [symbol] | [symbol] | | |
| [symbol] | [symbol] | similar to S. cereviase SSM4 | [symbol] | [symbol] | 102545.2 | [symbol] | [symbol] | N/A | | [symbol] | [symbol] | 21.8 | | [symbol] | [symbol] | 0 | |
| [symbol] | [symbol] | PREDICTED: similar to Vascular | [symbol] | [symbol] | 47909 | [symbol] | [symbol] | N/A | | [symbol] | [symbol] | | | [symbol] | [symbol] | | |
| | | endothelial growth factor receptor 1 | | | | | | | | | | | | | | | |
| | | precursor (VEGFR-1) (Vascular | | | | | | | | | | | | | | | |
| | | permeability factor receptor) (Tyroasine- | | | | | | | | | | | | | | | |
| | | protein kinase receptor FLT)(Flt-1) | | | | | | | | | | | | | | | |
| | | (Tyrosine-protein kinase FRT) (Fma-like | | | | | | | | | | | | | | | |
| [symbol] | [symbol] | zinc finger protein 639; Kruppel-like; zinc | [symbol] | [symbol] | 58054.4 | [symbol] | [symbol] | N/A | | [symbol] | [symbol] | | | [symbol] | [symbol] | | |
| | | finger protein ANC 2H01 | | | | | | | | | | | | | | | |
| [symbol] | [symbol] | Bent-like 1; BarH (Drosophila)-like 1 | [symbol] | [symbol] | 35074.5 | [symbol] | [symbol] | N/A | | [symbol] | [symbol] | 21.7 | | [symbol] | [symbol] | 5 | |
| [symbol] | [symbol] | calhepsin S preproprotein | [symbol] | [symbol] | 37495.7 | [symbol] | [symbol] | 1.02 | | [symbol] | [symbol] | 21.7 | | [symbol] | [symbol] | 2 | |
| [symbol] | [symbol] | sarco/endcoplasmic reticulum Ca2+- | [symbol] | [symbol] | 109258.2 | [symbol] | [symbol] | N/A | | [symbol] | [symbol] | | | [symbol] | [symbol] | | |
| | | ATPase isoform a; ATPase, Ca(2+)- | | | | | | | | | | | | | | | |
| | | transporting, ubiquitious; | | | | | | | | | | | | | | | |
| | | sarcoplasmic/endoplasmic reticulum | | | | | | | | | | | | | | | |
| | | calcium ATPase 3; SR Ca(2+)-ATPase 3; | | | | | | | | | | | | | | | |
| | | calcium pump 3; adenosine | | | | | | | | | | | | | | | |
| | | triphosphatase, calcium; | | | | | | | | | | | | | | | |
| [symbol] | [symbol] | glypican 5 | [symbol] | [symbol] | 53707 | [symbol] | [symbol] | N/A | | [symbol] | [symbol] | | | [symbol] | [symbol] | | |
| [symbol] | [symbol] | leucine rich repeat transmembrance | [symbol] | [symbol] | 59078.2 | [symbol] | [symbol] | 0.62 | | [symbol] | [symbol] | 21.5 | | [symbol] | [symbol] | 2 | |
| | | neuronal 2 | | | | | | | | | | | | | | | |
| [symbol] | [symbol] | solute carrier family 12, member 5; solute | [symbol] | [symbol] | 78224.8 | [symbol] | [symbol] | 0.94 | | [symbol] | [symbol] | 13.9 | | [symbol] | [symbol] | 1 | |
| | | carrier family 12 | | | | | | | | | | | | | | | |
| | | (sodium/potassium/chloride | | | | | | | | | | | | | | | |
| | | transporters), member 8; cation-chloride | | | | | | | | | | | | | | | |
| [symbol] | [symbol] | zinc finger protein 228 | [symbol] | [symbol] | 105076.4 | [symbol] | [symbol] | N/A | | [symbol] | [symbol] | 21.4 | | [symbol] | [symbol] | 1 | |
| [symbol] | [symbol] | PREDICTED: similar to Mucin 1 | [symbol] | [symbol] | 93058.4 | [symbol] | [symbol] | N/A | | [symbol] | [symbol] | 21.4 | | [symbol] | [symbol] | 0 | |
| | | Precursor (MUC-1) (Polymorphic | | | | | | | | | | | | | | | |
| | | epthelial mucin) (PEM) (PEMT) | | | | | | | | | | | | | | | |
| | | (Eplstalin) (Tumer-associated mucin) | | | | | | | | | | | | | | | |
| | | (Carcinoma-associated mucin) (Tumor- | | | | | | | | | | | | | | | |
| | | associated epithelial membrane antigen) | | | | | | | | | | | | | | | |
| | | (EMA) (H23AG) (Peanul-reactive urinary | | | | | | | | | | | | | | | |
| [symbol] | [symbol] | tankyrase, TRF1-interacting ankyrin- | [symbol] | [symbol] | 142011.5 | [symbol] | [symbol] | 1.38 | | [symbol] | [symbol] | 21.3 | | [symbol] | [symbol] | 0 | |
| | | related ADP-ribose polymerase | | | | | | | | | | | | | | | |
| [symbol] | [symbol] | 1-acylglycerol-3-phosphate O- | [symbol] | [symbol] | 43381 | [symbol] | [symbol] | 0.89 | | [symbol] | [symbol] | 21.3 | | [symbol] | [symbol] | 1 | |
| | | acyltransferase 3; lysophosphatidic acid | | | | | | | | | | | | | | | |
| | | acyltransferase-gamma1; 1-acyl-sn- | | | | | | | | | | | | | | | |
| | | glycerol-3-phosphate acyltransferase | | | | | | | | | | | | | | | |
| | | gamma; 1-AGP acyltransferase 3 | | | | | | | | | | | | | | | |
| [symbol] | [symbol] | ATP-binding cassette, sub-family C, | [symbol] | [symbol] | 149540.8 | [symbol] | [symbol] | 0.57 | | [symbol] | [symbol] | 21.3 | | [symbol] | [symbol] | 0 | |
| | | member 4; canallcular multispecific | | | | | | | | | | | | | | | |
| | | organic anion transporter (ABC | | | | | | | | | | | | | | | |
| [symbol] | [symbol] | brain glycogen phosphorylase; glycogen | [symbol] | [symbol] | 96696 | [symbol] | [symbol] | N/A | | [symbol] | [symbol] | 21.3 | | [symbol] | [symbol] | 1 | |
| | | phosphorylase B | | | | | | | | | | | | | | | |
| [symbol] | [symbol] | potassium channel tetramerisation | [symbol] | [symbol] | 88984 | [symbol] | [symbol] | N/A | | [symbol] | [symbol] | 14.6 | | [symbol] | [symbol] | 1 | |
| | | domain containing 3; NY-REN-45 antigen | | | | | | | | | | | | | | | |
| [symbol] | [symbol] | PREDICTED: similar to ODZ3 | [symbol] | [symbol] | 21695 | [symbol] | [symbol] | 0.99 | | [symbol] | [symbol] | 21.3 | | [symbol] | [symbol] | 8 | |
| [symbol] | [symbol] | potassium voltage-gated channel, Shal- | [symbol] | [symbol] | 70536.5 | [symbol] | [symbol] | N/A | | [symbol] | [symbol] | | | [symbol] | [symbol] | | |
| | | related subfamily, member 2; voltage- | | | | | | | | | | | | | | | |
| | | sensitive potassium channel; voltage- | | | | | | | | | | | | | | | |
| | | gated potassium channel Kv4.2 | | | | | | | | | | | | | | | |
| [symbol] | [symbol] | PlggyBac transpossble element derived 2 | [symbol] | [symbol] | 68011.4 | [symbol] | [symbol] | 0.68 | | [symbol] | [symbol] | 21.1 | | [symbol] | [symbol] | 2 | |
| [symbol] | [symbol] | hypothetical protein FLI90430 | [symbol] | [symbol] | 61740.9 | [symbol] | [symbol] | 0.79 | | [symbol] | [symbol] | 11.8 | | [symbol] | [symbol] | 1 | |
| [symbol] | [symbol] | alyl hydrocarbon receptor | [symbol] | [symbol] | 96147.4 | [symbol] | [symbol] | 0.91 | | [symbol] | [symbol] | 13.5 | | [symbol] | [symbol] | 0 | |
| [symbol] | [symbol] | SH3 and cysleine rich domain; arc | [symbol] | [symbol] | 44553.5 | [symbol] | [symbol] | N/A | | [symbol] | [symbol] | 21 | | [symbol] | [symbol] | 2 | |
| | | homology three (SH3) and cysleine rich | | | | | | | | | | | | | | | |
| | | domain | | | | | | | | | | | | | | | |
| [symbol] | [symbol] | phosphatidylinoskol-4-phosphate 5- | [symbol] | [symbol] | 61036.3 | [symbol] | [symbol] | 0.49 | | [symbol] | [symbol] | 20.9 | | [symbol] | [symbol] | 1 | |
| | | kinase, type I, beta | | | | | | | | | | | | | | | |
| [symbol] | [symbol] | laucine-rich repeats and immumoglobulin- | [symbol] | [symbol] | 76433.8 | [symbol] | [symbol] | N/A | | [symbol] | [symbol] | | | [symbol] | [symbol] | | |
| | | like domains 4 | | | | | | | | | | | | | | | |
| [symbol] | [symbol] | tripartile motif-containing 3g isoform 1; | [symbol] | [symbol] | 59990.4 | [symbol] | [symbol] | N/A | | [symbol] | [symbol] | | | [symbol] | [symbol] | | |
| | | ring finger protein 23; testis-abundent | | | | | | | | | | | | | | | |
| | | finger protein | | | | | | | | | | | | | | | |
| [symbol] | [symbol] | hypothetical protein FLJ23153 | [symbol] | [symbol] | 31814.1 | [symbol] | [symbol] | 1.02 | | [symbol] | [symbol] | 20.8 | | [symbol] | [symbol] | 3 | |
| [symbol] | [symbol] | dihydropyrimidinase-like 4 | [symbol] | [symbol] | 61905.7 | [symbol] | [symbol] | N/A | | [symbol] | [symbol] | | | [symbol] | [symbol] | | |
| [symbol] | [symbol] | growth hormone 2 isoform 3; hGH-V | [symbol] | [symbol] | 27101.2 | [symbol] | [symbol] | 1.05 | | [symbol] | [symbol] | 20.6 | | [symbol] | [symbol] | 3 | |
| | | placental-specific growth hormone; | | | | | | | | | | | | | | | |
| | | placenta-specific growth hormone | | | | | | | | | | | | | | | |
| [symbol] | [symbol] | KIAA0218 gene product | [symbol] | [symbol] | 85023.1 | [symbol] | [symbol] | 0.89 | | [symbol] | [symbol] | 12.8 | | [symbol] | [symbol] | 2 | |
| [symbol] | [symbol] | olfactory receptor, family 6, subfamily C, | [symbol] | [symbol] | 35116.5 | [symbol] | [symbol] | 1.09 | | [symbol] | [symbol] | 20.5 | | [symbol] | [symbol] | 4 | |
| | | member 76 | | | | | | | | | | | | | | | |
| [symbol] | [symbol] | coli division cycle 2-like 5 isoform 1; | [symbol] | [symbol] | 164970.3 | [symbol] | [symbol] | N/A | | [symbol] | [symbol] | 20.3 | | [symbol] | [symbol] | 0 | |
| | | CDC2-related protein kinase 5 | | | | | | | | | | | | | | | |
| [symbol] | [symbol] | hypothetical protein FLJ38808 | [symbol] | [symbol] | 64517.4 | [symbol] | [symbol] | N/A | | [symbol] | [symbol] | | | [symbol] | [symbol] | | |
| [symbol] | [symbol] | phosphofurin acidic cluster sorting | [symbol] | [symbol] | 104898.4 | [symbol] | [symbol] | N/A | | [symbol] | [symbol] | | | [symbol] | [symbol] | | |
| | | protein 1; cytosolic sorting protein PAC8- | | | | | | | | | | | | | | | |
| [symbol] | [symbol] | sushi domain containing 2; Sushi domain | [symbol] | [symbol] | 90207.7 | [symbol] | [symbol] | N/A | | [symbol] | [symbol] | 20.3 | | [symbol] | [symbol] | 1 | |
| | | (SCR repeat) containing | | | | | | | | | | | | | | | |
| [symbol] | [symbol] | myosin XV; unconvertional myosin-15 | [symbol] | [symbol] | 395219.5 | [symbol] | [symbol] | N/A | | [symbol] | [symbol] | 20.2 | | [symbol] | [symbol] | 0 | |
| [symbol] | [symbol] | chamokine (C—C motif) receptor 8; | [symbol] | [symbol] | 40844.4 | [symbol] | [symbol] | N/A | | [symbol] | [symbol] | 20.2 | | [symbol] | [symbol] | 2 | |
| | | chamokine (C—C) receptor 8; chamokine | | | | | | | | | | | | | | | |
| | | (C—C) receptor-like 2; CC-chamokine | | | | | | | | | | | | | | | |
| | | receptor chamr1 | | | | | | | | | | | | | | | |
| [symbol] | [symbol] | PERQ amino acid rich with GYF domain | [symbol] | [symbol] | 89740.9 | [symbol] | [symbol] | N/A | | [symbol] | [symbol] | 20.2 | | [symbol] | [symbol] | 1 | |
| | | 1; postmelotic segregation Increased 2- | | | | | | | | | | | | | | | |
| | | like 12; Grb10 interacting GYF protein 1 | | | | | | | | | | | | | | | |
| [symbol] | [symbol] | vacuolar protein sorting 29 isoform 1; | [symbol] | [symbol] | 20505.7 | [symbol] | [symbol] | 0.72 | | [symbol] | [symbol] | 20.2 | | [symbol] | [symbol] | 3 | |
| | | vacuolar sorting protein VPS29/PEP11; | | | | | | | | | | | | | | | |
| | | vacuolar protein sorting 29 (yeast | | | | | | | | | | | | | | | |
| | | homolog); retromer protein; x007 protein | | | | | | | | | | | | | | | |
| [symbol] | [symbol] | hypothetical protein MGC45888 | [symbol] | [symbol] | 31064.9 | [symbol] | [symbol] | N/A | | [symbol] | [symbol] | 20.1 | | [symbol] | [symbol] | 5 | |
| [symbol] | [symbol] | D site of albumin promoter (albumin D- | [symbol] | [symbol] | 34348.9 | [symbol] | [symbol] | 0.57 | | [symbol] | [symbol] | | | [symbol] | [symbol] | | |
| | | box) binding protein; D site of albumin | | | | | | | | | | | | | | | |
| | | promoter binding protein | | | | | | | | | | | | | | | |
| [symbol] | [symbol] | transducin-like enhancer protein 2; | [symbol] | [symbol] | 79341 | [symbol] | [symbol] | 0.8 | | [symbol] | [symbol] | 20.1 | | [symbol] | [symbol] | 2 | |
| | | transducin-like enhancer of split 2; | | | | | | | | | | | | | | | |
| | | enhancer of split groucho 2; transducin- | | | | | | | | | | | | | | | |
| | | like enhancer of split 2, homolog of | | | | | | | | | | | | | | | |
| | | Drosophila E(sp1) | | | | | | | | | | | | | | | |
| [symbol] | [symbol] | hypothetical gene MGC 16309 | [symbol] | [symbol] | 36338.8 | [symbol] | [symbol] | 0.61 | | [symbol] | [symbol] | | | [symbol] | [symbol] | | |
| [symbol] | [symbol] | splicing factor, arginineserine-rich 1 | [symbol] | [symbol] | 27744.6 | [symbol] | [symbol] | 1.79 | | [symbol] | [symbol] | | | [symbol] | [symbol] | | |
| | | (splicing factor 2, alternate splicing | | | | | | | | | | | | | | | |
| [symbol] | [symbol] | super conserved receptor expressed in | [symbol] | [symbol] | 41481.4 | [symbol] | [symbol] | N/A | | [symbol] | [symbol] | 20 | | [symbol] | [symbol] | 5 | |
| | | brain 3 | | | | | | | | | | | | | | | |
| [symbol] | [symbol] | hypothetical protein XP, 211108 | [symbol] | [symbol] | 10737.5 | [symbol] | [symbol] | 1.41 | | [symbol] | [symbol] | 20 | | [symbol] | [symbol] | 17 | |

According to these results, 158 proteins could be identified and comparatively quantified when analyzing this fraction (SCX 50 fraction) on ABI-4700 and selecting, at Rank 1, peptides having a Mascot score of 30 or higher, and about 286 proteins were identified and comparatively quantified when selecting peptides a Peptide Score of 20 or higher. When the SCX 50 fraction was analyzed similarly in the C18-nanoLC/Q-Star system, 119 proteins could be identified and quantified in the case of selecting, at Rank 1, peptides having a Peptide Score of 20 or higher. In addition, the ratios of H/L-chain labeling (comparative quantification values) of most proteins were approximately 1, and thus it appears that the comparative quantification method according to the present improvement can be satisfactory.

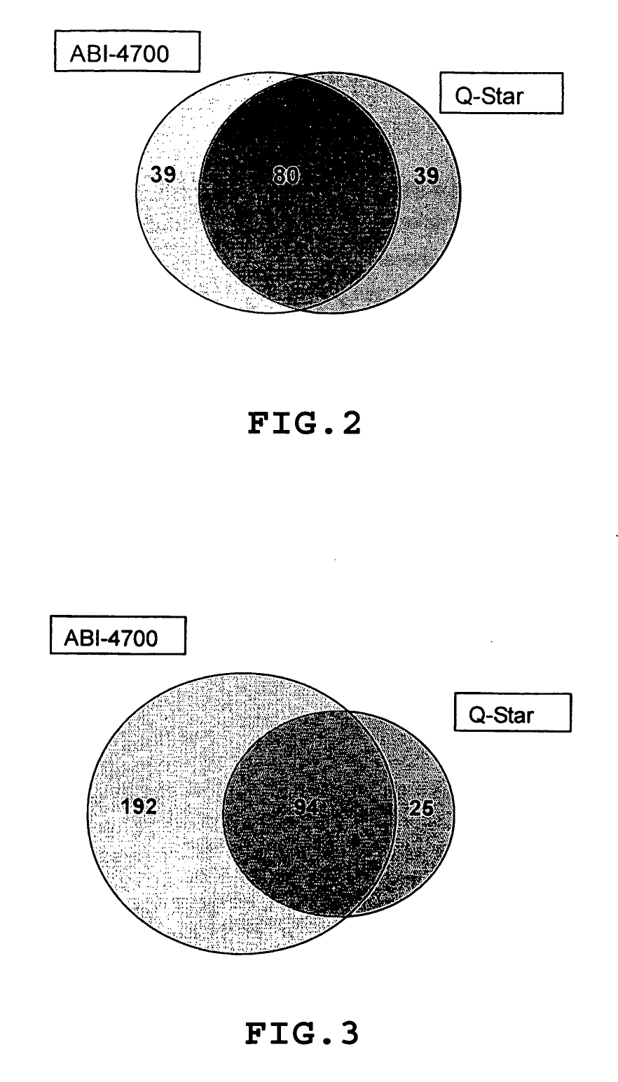

By comparing top 119 proteins on ABI-4700 and top 119 proteins on Q-Star, 80 proteins were common in both, 39 proteins were determined and quantified only on Q-Star, 39 proteins only on ABI-4700, and a total of 158 proteins on either of the instruments (FIG. 2). When selecting a score of 20 or higher on ABI-4700 and on Q-Star, 94 proteins were common in both, 25 proteins were identified only on Q-Star, 192 proteins only on ABI-4700, and a total of 311 proteins on either of the instruments (FIG. 3).

It turns out from the above-described results that by using the method according to the present invention, a plurality of small-amount proteins in serum can be identified and comparatively quantified.

### Comparative Example

### Identification and Quantification of Serum Proteins by Conventional Method

According to the routine procedure, serum (in which the six major proteins, including albumin, had been removed) was reacted with a cICAT reagent, the resultant labeled proteins were digested with trypsin, and the reaction solution containing the trypsin digestion products was loaded onto SCX column chromatography to thoroughly remove reagent-derived substances and others, followed by fractionating the peptide fraction into 50 sub-fractions with a salt concentration gradient method. The obtained sub-fractions were further loaded onto an avidin affinity column to specifically purify labeled peptides containing biotin. The labeled peptides containing biotin were treated with TFA to cleave the biotin segment and others, followed by evaporation to dryness. The obtained samples were subjected to measurements on a mass spectrometer to identify and quantify serum proteins, whereby major serum proteins (30 to 50 proteins, Mascot Scores of 20 or higher) could be identified and quantified, small-amount proteins could hardly be identified. From the results of the investigation as to this cause, it turned out that each of the fractionated samples after the above-described TFA treatment contains biotin at a much larger amount than the equivalent amount of biotin derived from the labeled peptides containing biotin.

What is claimed is:

1. A method for the analysis of differential expression of proteins employing an isotope label, characterized by cleaving a tag from peptides labeled with an ICAT reagent containing a cleavable tag, separating and purifying the resultant labeled peptides, and performing an analysis in mass spectrometry.

2. The method according to claim 1, wherein the step of separation and purification is carried out using column chromatography and wherein the removal of the tag and others the separation and purification of the cICAT-labeled peptides are carried out concurrently.

3. The method according to claim 1 or 2, wherein the tag is biotin.

4. The method according to any one of claims 1 to 3, wherein the peptides are derived from a serum protein.

5. A system for the analysis of differential expression of small-amount proteins in a sample, characterized by employing a method according to any one of claims 1 to 3.

6. The system according to claim 5, wherein the sample is a serum sample.
